(19) United States
(12) Patent Application Publication
(10) Pub. No.: US 2021/0030703 A1
(43) Pub. Date: Feb. 4, 2021

(54) USE OF CALORIC RESTRICTION MIMETICS FOR POTENTIATING CHEMO-IMMUNOTHERAPY FOR THE TREATMENT OF CANCERS

(71) Applicants: INSERM (INSTITUT NATIONAL DE LA SANTÉ ET DE LA RECHERCHE MÉDICALE), Paris (FR); UNIVERSITÉ DE PARIS, Paris (FR); SORBONNE UNIVERSITÉ, Paris (FR); ASSISTANCE PUBLIQUE-HÔPITAUX DE PARIS (APHP), Paris (FR); UNIVERSITÉ PARIS-SACLAY, Saint Aubin (FR)

(72) Inventors: Guido KROEMER, Paris cedex 06 (FR); Sarah LEVESQUE, Paris (FR); Jonathan POL, Paris (FR)

(73) Assignees: INSERM (INSTITUT NATIONAL DE LA SANTÉ ET DE LA RECHERCHE MÉDICALE), Paris (FR); UNIVERSITÉ DE PARIS, Paris (FR); SORBONNE UNIVERSITÉ, Paris (FR); ASSISTANCE PUBLIQUE-HÔPITAUX DE PARIS (APHP), Paris (FR); UNIVERSITÉ PARIS-SACLAY, Saint Aubin (FR)

(21) Appl. No.: 16/980,003

(22) PCT Filed: Mar. 11, 2019
(86) PCT No.: PCT/EP2019/056041

§ 371 Date: Sep. 11, 2020

(30) Foreign Application Priority Data
Mar. 12, 2018 (EP) 18305257.0
Mar. 29, 2018 (EP) 18305359.4

(51) Int. Cl. A61K 31/194; C07K 16/28; A61K 31/385; A61P 35/00

(52) U.S. Cl. CPC A61K 31/194; A61P 35/00; A61K 31/385; C07K 16/2818

(57) ABSTRACT

In most cases, cancer chemotherapy and immunotherapy fail to yield durable responses, and complete and permanent regression of established tumors are rare. Here the inventors show that so-called caloric restriction mimetics (CRMs), which are natural or synthetic compounds that pharmacologically mimic the effects of fasting or caloric restriction, can be used to enhance the probability of cancer cure. The administration of several chemically distinct CRMs (such as hydroxycitrate, lipoic acid and the natural polyamine spermidine) led to the complete regression and the induction of protective anticancer immune responses in mouse models. This effect was achieved when CRMs were combined with chemotherapy and immunotherapy targeting the immune checkpoint molecules CTLA-4 and/or PD-1. Hence, caloric restriction and CRMs can be used to sensitize cancers to chemo-immunotherapy.

### FIELD OF THE INVENTION

The present invention relates to the use of caloric restriction mimetics for potentiating chemo-immunotherapy for the treatment of cancers.

### BACKGROUND OF THE INVENTION

Caloric restriction and fasting constitute efficient dietary manipulations to induce autophagy and to mediate positive effects on organismal health. Caloric restriction mimetics (CRMs) are compounds that mimic the biochemical and physiological consequences of caloric restriction and fasting. CRMs stimulate autophagy by favouring the deacetylation of cellular proteins, mostly in the cytoplasm of the cell. This deacetylation process can be achieved by three classes of compounds that (i) deplete the cytosolic pool of acetyl coenzyme A (AcCoA; the sole donor of acetyl groups), (ii) inhibit acetyl transferases (a group of enzymes that acetylate lysine residues in an array of proteins) or (iii) that stimulate the activity of deacetylases and hence reverse the action of acetyl transferases.(1) Examples for the first class of CRMs (compounds that deplete AcCoA) include inhibitors of the ATP citrate lyase (ACLY) such as hydroxycitrate (HC) and SB204990, but also agents that inhibit upstream reactions leading to the formation of AcCoA as a final result of glycolysis, amino acid catabolism or fatty acid oxidation.(2) Examples for the second class of CRMs (compounds that inhibit acetyltransferases) include inhibitors of the enzymatic activity of EP300 including, but not limited to, anacardic acid, salicylate and salicylate derivatives, epigallocatechine gallate (EGCG), spermidine and the compound C646.(3, 4) Examples for the third class of CRMs (compounds that activate deacetylases) include resveratrol and synthetic agents such as SRT1720 that activate sirtuin-1, a major deacetylase that efficiently deacetylates proteins that have been acetylated by EP300.(5-8). It was shown that agents falling into each of the three classes of CRMs (for class I: HC and SB2049901; for class II: spermidine and C646; for class III: resveratrol) are able to improve the efficacy of anticancer chemotherapy with immunogenic cell death (ICD) inducers.(9)

Immunogenic cell death (ICD) inducers are pharmacological compounds that kill malignant cells in a way that they elicit an anticancer immune response.(10-19) This is linked to the induction of premortem stress pathways (such as autophagy, endoplasmic reticulum stress, type-1 interferon response) and the release or surface exposure of multiple danger-associated molecular patterns (DAMPs) including, but not limited to, adenosine triphosphate (ATP), annexin A1 (ANXA1), calreticulin (CALR), high mobility group protein B1 (HMGB1), type-1 interferons and chemokines. These DAMPs act on pattern recognition receptors (PRRs) that include, but are not limited to, purinergic receptors (mostly P2Y2 and P2X7 for ATP), formyl peptide receptor 1 (FPR1 for ANXA1), CD91 (for CALR), TLR4 (for HMGB1), type-1 interferon receptor (IFNAR) and chemokine receptors that are mostly expressed by myeloid cells, including dendritic cells (DCs) and their precursors. In essence, the DAMPs released as a consequence of ICD engage PRRs to attract DC precursors into tumor bed (as a result of the ATP-P2Y2 interaction), cause them to juxtapose to dying cancer cells (as a result of the interaction between ANXA1 and FPR1), transfer dead-cell antigens from tumor cells to DC precursors (as a result of the CALR-CD91 interaction), the maturation of DCs so that they can cross-present tumor-associated antigens (as a result of the HMGB1-TLR4 interaction), hence eliciting a cellular immune response that requires the recruitment of T lymphocytes into the tumor bed (as a result of the interaction of chemokines with their receptors).(10-19) There is preclinical and clinical evidence indicating that chemotherapeutics that induce ICD include anthracyclines, oxaliplatin and taxanes, as well radiotherapy (that can induce ICD), mediate their long-term antineoplastic effects via the stimulation of an anticancer immune response.(10, 20-23) The mechanisms through which CRMs potentiate the efficacy of ICD inducers are immune-mediated. In other words, depletion of CD8⁺ T lymphocytes results in the abolition of the combination effect.(9) Apparently, CRMs stimulate autophagy in malignant cells (and presumably also in other cell types including immune cells) and this boosts the anticancer immune response.(1, 2, 9)

Immune checkpoint inhibitors (ICIs) have revolutionized the treatment of cancer over the past few years.(24-26) Thus, antibodies that block CTLA-4 or the interaction between PD-1 and PD-L1 are widely used in the modern oncological armamentarium for a wide range of different cancer types. It is expected that new indications for such ICIs, alone or in combination with other therapeutic agents, will enter clinical routine soon. Moreover, novel ICIs are being developed. As a general principle, ICIs subvert immunosuppressive circuitries with the final result of reactivating the anticancer immune response. Nevertheless, not all tumors are responsive to ICI-mediated therapy.

However, the combination of chemotherapy and/or immunotherapy with caloric restriction mimetics has never been investigated. It was evidenced by the inventors that starvation or CRM therapy as such does not have an effect of tumor sensibility towards ICI immunotherapy. The experiments carried-out in the context of the present invention confirm that chemotherapy, enhances tumor sensibility towards ICI immunotherapy. Yet, it was surprisingly found by the Applicants that the association of CRMs along with chemotherapy, not only render tumor cells significantly responsive to ICI immune-therapy, but even more surprisingly exert a significant inhibition in tumor cell growth.

Advantageously, the association of CRMs, chemotherapy and ICIs according to the present invention established a long-lasting cancer-specific memory and thus impeding cancer recurrence in the treated subject.

### SUMMARY OF THE INVENTION

The present invention relates to a method of treating cancer in a patient in need thereof comprising administering to the patient a therapeutically effective combination of chemotherapy and an immune checkpoint inhibitor with a caloric restriction mimetic. In particular, the present invention is defined by the claims.

### DETAILED DESCRIPTION OF THE INVENTION

In most cases, cancer chemotherapy and immunotherapy fail to yield durable responses, and complete and permanent regression of established tumors are rare. Here the inventors show that so-called caloric restriction mimetics (CRMs), which are natural or synthetic compounds that pharmacologically mimic the effects of fasting or caloric restriction, can be used to enhance the probability of cancer cure. The administration of several chemically distinct CRMs (exemplified by the salicylate aspirin, the citrate derivative hydroxycitrate and the natural polyamine spermidine) led to the complete regression and the induction of protective anticancer immune responses in mouse models. These effects were achieved when CRMs were combined with chemotherapy and immunotherapy targeting the immune checkpoint molecules CTLA-4 and/or PD-1. The inventors also show that the blockade of the CD11b-dependent extravasation of myeloid cells blocks such a combination effect as well. Hence, caloric restriction and CRMs can be used to sensitize cancers to chemo-immunotherapy.

Accordingly, the first object of the present invention relates to a method of treating cancer in a patient in need thereof comprising administering to the patient a therapeutically effective combination of chemotherapy and/or immunotherapy with a caloric restriction mimetic.

The present invention also relates to a method of treating cancer in a patient in need thereof comprising administering to the patient a therapeutically effective combination of chemotherapy and immunotherapy with a caloric restriction mimetic, wherein the chemotherapy, the immunotherapy and the caloric restriction mimetic are administered separately.

As used herein, the term “subject”, “individual,” or “patient” is used interchangeably and refers to any subject for whom diagnosis, treatment, or therapy is desired, particularly humans. Other subjects may include cattle, dogs, cats, guinea pigs, rabbits, rats, mice, horses, and the like. In some preferred embodiments, the subject is a human. In one embodiment, the subject is a subjected to a first-line cancer therapy. In one embodiment, the subject is subjected to a second-line cancer therapy. In one embodiment, the subject is not responsive to a first-line or a second-line cancer therapy. In one embodiment, the patient is a geriatric patient.

In one embodiment, the patient had been previously subjected to radiotherapy. In one embodiment, the patient had been previously subjected a surgical removal of a tumor.

As used herein, the term “cancer” has its general meaning in the art and includes, but is not limited to, solid tumors and blood-borne tumors. The term cancer includes malignant diseases of any tissues/organs. The term “cancer” further encompasses both primary and metastatic cancers. Examples of cancers that may be treated by methods and compositions of the invention include, but are not limited to, cancer cells from the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some embodiments, the method of the present invention is particularly suitable for the treatment of triple negative breast cancer. As used herein, the term“triple negative breast cancer” refers to those breast cancer cells that are negative for estrogen (ER), progesterone (PR) and HER2/neu (HER2) receptors. The “triple negative” status for breast cancer cells is generally associated with a poor prognosis in early breast cancer patients. The term “triple negative breast cancer” is often used interchangeably or as a clinical surrogate for “basal-like” breast cancers.

In one embodiment the cancer is of high recurrence. In one embodiment the cancer is a recurrent cancer past surgery removal and/or radiotherapy. In one embodiment, the cancer is not responding to a first or second line chemotherapy.

In one embodiment the cancer is selected from autophagy compent carcinomas. Autophagy designates the catabolic process involving the degradation of a cell's own components; such as, long lived proteins, protein aggregates, cellular organelles, cell membranes, organelle membranes, and other cellular components. The mechanism of autophagy may include: (i) the formation of a membrane around a targeted region of the cell, separating the contents from the rest of the cytoplasm, (ii) the fusion of the resultant vesicle with a lysosome and the subsequent degradation of the vesicle contents. The term autophagy may also refer to one of the mechanisms by which a starving cell re-allocates nutrients from unnecessary processes to more essential processes.

In one embodiment the cancer is a cancer not responding to immunotherapy, namely ICI immune therapy.

In one embodiment the cancer is selected from pancreas carcinoma, stomach carcinoma, adenocarcinoma, colon carcinoma, rectal carcinoma, adenocarcinoma, glioma, glioblastoma and lung cancer, preferably non-small cell lung cancer.

In one embodiment the cancer is selected from pancreas carcinoma, glioma, glioblastoma and lung cancer, preferably non-small cell lung cancer. In one embodiment the cancer is selected from glioma, glioblastoma and lung cancer, preferably non-small cell lung cancer. In one embodiment the cancer is selected from glioma and glioblastoma.

In one embodiment the cancer is selected from lung cancer, preferably non-small cell lung cancer. In particular, the method of the present invention is particularly suitable for the treatment of cancer characterized by a low tumor infiltration of CD8+ T cells.

As used herein, the term “CD8+ T cell” has its general meaning in the art and refers to a subset of T cells which express CD8 on their surface. They are MHC class I-restricted, and function as cytotoxic T cells. “CD8+ T cells” are also called cytotoxic T lymphocytes (CTL), T-killer cells, cytolytic T cells, or killer T cells. CD8 antigens are members of the immunoglobulin supergene family and are associative recognition elements in major histocompatibility complex class I-restricted interactions.

As used herein, the term “tumor infiltrating CD8+ T cell” refers to the pool of CD8+ T cells of the patient that have left the blood stream and have migrated into a tumor.

Typically said tumor-inflitration of CD8+ T cells is determined by any convention method in the art. For example, said determination comprises quantifying the density of CD8+ T cells in a tumor sample obtained from the patient.

As used herein, the term “tumor tissue sample” means any tissue tumor sample derived from the patient. Said tissue sample is obtained for the purpose of the in vitro evaluation. In some embodiments, the tumor sample may result from the tumor resected from the patient. In some embodiments, the tumor sample may result from a biopsy performed in the primary tumor of the patient or performed in metastatic sample distant from the primary tumor of the patient. For example an endoscopical biopsy performed in the bowel of the patient affected by a colorectal cancer. In some embodiments, the tumor tissue sample encompasses (i) a global primary tumor (as a whole), (ii) a tissue sample from the center of the tumor, (iii) a tissue sample from the tissue directly surrounding the tumor which tissue may be more specifically named the “invasive margin” of the tumor, (iv) lymphoid islets in close proximity with the tumor, (v) the lymph nodes located at the closest proximity of the tumor, (vi) a tumor tissue sample collected prior surgery (for follow-up of patients after treatment for example), and (vii) a distant metastasis. As used herein the “invasive margin” has its general meaning in the art and refers to the cellular environment surrounding the tumor. In some embodiments, the tumor tissue sample, irrespective of whether it is derived from the center of the tumor, from the invasive margin of the tumor, or from the closest lymph nodes, encompasses pieces or slices of tissue that have been removed from the tumor center of from the invasive margin surrounding the tumor, including following a surgical tumor resection or following the collection of a tissue sample for biopsy, for further quantification of one or several biological markers, notably through histology or immunohistochemistry methods, through flow cytometry methods and through methods of gene or protein expression analysis, including genomic and proteomic analysis. The tumor tissue sample can, of course, be subjected to a variety of well-known post-collection preparative and storage techniques (e.g., fixation, storage, freezing, etc.). The sample can be fresh, frozen, fixed (e.g., formalin fixed), or embedded (e.g., paraffin embedded).

In some embodiments, the quantification of density of CD8+ T cells is determined by immunohistochemistry (IHC). For example, the quantification of the density of CD8+ T cells is performed by contacting the tissue tumor tissue sample with a binding partner (e.g. an antibody) specific for a cell surface marker of said cells. Typically, the quantification of density of CD8+ T cells is performed by contacting the tissue tumor tissue sample with a binding partner (e.g. an antibody) specific for CD8. Typically, the density of CD8+ T cells is expressed as the number of these cells that are counted per one unit of surface area of tissue sample, e.g. as the number of cells that are counted per cm² or mm² of surface area of tumor tissue sample. In some embodiments, the density of cells may also be expressed as the number of cells per one volume unit of sample, e.g. as the number of cells per cm3 of tumor tissue sample. In some embodiments, the density of cells may also consist of the percentage of the specific cells per total cells (set at 100%) Immunohistochemistry typically includes the following steps i) fixing the tumor tissue sample with formalin, ii) embedding said tumor tissue sample in paraffin, iii) cutting said tumor tissue sample into sections for staining, iv) incubating said sections with the binding partner specific for the marker, v) rinsing said sections, vi) incubating said section with a secondary antibody typically biotinylated and vii) revealing the antigen-antibody complex typically with avidin-biotin-peroxidase complex. Accordingly, the tumor tissue sample is firstly incubated the binding partners. After washing, the labeled antibodies that are bound to marker of interest are revealed by the appropriate technique, depending of the kind of label is borne by the labeled antibody, e.g. radioactive, fluorescent or enzyme label. Multiple labeling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. H&E, DAPI, Hoechst. Other staining methods may be accomplished using any suitable method or system as would be apparent to one of skill in the art, including automated, semi-automated or manual systems. For example, one or more labels can be attached to the antibody, thereby permitting detection of the target protein (i.e the marker). Exemplary labels include radioactive isotopes, fluorophores, ligands, chemiluminescent agents, enzymes, and combinations thereof. In some embodiments, the label is a quantum dot. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. ³H, ¹⁴C, ³²P, ³⁵S or ¹²⁵I) and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In other examples, the antibody can be conjugated to peptides or proteins that can be detected via a labeled binding partner or antibody. In an indirect IHC assay, a secondary antibody or second binding partner is necessary to detect the binding of the first binding partner, as it is not labeled. The resulting stained specimens are each imaged using a system for viewing the detectable signal and acquiring an image, such as a digital image of the staining Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the marker in the sample. Various automated sample processing, scanning and analysis systems suitable for use with immunohistochemistry are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample. Quantitative or semi-quantitative histochemistry refers to method of scanning and scoring samples that have undergone histochemistry, to identify and quantitate the presence of the specified biomarker (i.e. the marker). Quantitative or semi-quantitative methods can employ imaging software to detect staining densities or amount of staining or methods of detecting staining by the human eye, where a trained operator ranks results numerically. For example, images can be quantitatively analyzed using a pixel count algorithms (e.g., Aperio Spectrum Software, Automated QUantitatative Analysis platform (AQUA® platform), and other standard methods that measure or quantitate or semi-quantitate the degree of staining; see e.g., U.S. Pat. Nos. 8,023,714; 7,257,268; 7,219,016; 7,646,905; published U.S. Patent Publication No. US20100136549 and 20110111435; Camp et al. (2002) Nature Medicine, 8:1323-1327; Bacus et al. (1997) Analyt Quant Cytol Histol, 19:316-328). A ratio of strong positive stain (such as brown stain) to the sum of total stained area can be calculated and scored. The amount of the detected biomarker (i.e. the marker) is quantified and given as a percentage of positive pixels and/or a score. For example, the amount can be quantified as a percentage of positive pixels. In some examples, the amount is quantified as the percentage of area stained, e.g., the percentage of positive pixels. For example, a sample can have at least or about at least or about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95% or more positive pixels as compared to the total staining area. In some embodiments, a score is given to the sample that is a numerical representation of the intensity or amount of the histochemical staining of the sample, and represents the amount of target biomarker (e.g., the marker) present in the sample. Optical density or percentage area values can be given a scaled score, for example on an integer scale. Thus, in some embodiments, the method of the present invention comprises the steps consisting in i) providing one or more immunostained slices of tissue section obtained by an automated slide-staining system by using a binding partner capable of selectively interacting with the marker (e.g. an antibody as above described), ii) proceeding to digitalisation of the slides of step a. by high resolution scan capture, iii) detecting the slice of tissue section on the digital picture iv) providing a size reference grid with uniformly distributed units having a same surface, said grid being adapted to the size of the tissue section to be analyzed, and v) detecting, quantifying and measuring intensity of stained cells in each unit whereby the number or the density of cells stained of each unit is assessed.

In some embodiments, the cell density of CD8+ T cells is determined in the whole tumor tissue sample, is determined in the invasive margin or centre of the tumor tissue sample or is determined both in the centre and the invasive margin of the tumor tissue sample.

Accordingly a further object of the present invention relates to a method of treating cancer in a patient in need thereof comprising i) quantifying the density of CD8+ T cells in a tumor tissue sample obtained from the patient ii) comparing the density quantified at step i) with a predetermined reference value and iii) administering to the patient a therapeutically effective combination of chemotherapy and immunotherapy with the caloric restriction mimetic when the density determined at step i) is lower than the predetermined value.

In some embodiments, the predetermined value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of cell densities in properly banked historical patient samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after quantifying the density of CD8+ T cells in a group of reference, one can use algorithmic analysis for the statistic treatment of the measured densities in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the predetermined reference value correlates with the survival time of the patient. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they've become cancer-free (achieved remission). DSF gives more specific information and is the number of people with a particular cancer who achieve remission. Also, progression-free survival (PFS) rates (the number of people who still have cancer, but their disease does not progress) includes people who may have had some success with treatment, but the cancer has not disappeared completely. As used herein, the expression “short survival time” indicates that the patient will have a survival time that will be lower than the median (or mean) observed in the general population of patients suffering from said cancer. When the patient will have a short survival time, it is meant that the patient will have a “poor prognosis”. Inversely, the expression “long survival time” indicates that the patient will have a survival time that will be higher than the median (or mean) observed in the general population of patients suffering from said cancer. When the patient will have a long survival time, it is meant that the patient will have a “good prognosis”. In some embodiments, the predetermined reference value is determined by carrying out a method comprising the steps of a) providing a collection of tumor tissue samples from patient suffering from the cancer of interest; b) providing, for each tumor tissue sample provided at step a), information relating to the actual clinical outcome for the corresponding patient (i.e. the duration of the disease-free survival (DFS) and/or the overall survival (OS)); c) providing a serial of arbitrary quantification values; d) quantifying the density of CD8+ T cells for each tumor tissue sample contained in the collection provided at step a); e) classifying said tumor tissue samples in two groups for one specific arbitrary quantification value provided at step c), respectively: (i) a first group comprising tumor tissue samples that exhibit a quantification value for level that is lower than the said arbitrary quantification value contained in the said serial of quantification values; (ii) a second group comprising tumor tissue samples that exhibit a quantification value for said level that is higher than the said arbitrary quantification value contained in the said serial of quantification values; whereby two groups of tumor tissue samples are obtained for the said specific quantification value, wherein the tumor tissue samples of each group are separately enumerated; f) calculating the statistical significance between (i) the quantification value obtained at step e) and (ii) the actual clinical outcome of the patients from which tumor tissue samples contained in the first and second groups defined at step f) derive; g) reiterating steps f) and g) until every arbitrary quantification value provided at step d) is tested; h) setting the said predetermined reference value as consisting of the arbitrary quantification value for which the highest statistical significance (most significant) has been calculated at step g). For example, the density of CD8+ T cells has been assessed for 100 tumor tissue samples of 100 patients. The 100 samples are ranked according to the density of CD8+ T cells. Sample 1 has the highest density and sample 100 has the lowest density. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer patient, Kaplan Meier curves are prepared for each of the 99 groups of two subsets. Also for each of the 99 groups, the p value between both subsets was calculated. The predetermined reference value is then selected such as the discrimination based on the criterion of the minimum p value is the strongest. In other terms, the density of CD8+ T cells corresponding to the boundary between both subsets for which the p value is minimum is considered as the predetermined reference value. It should be noted that the predetermined reference value is not necessarily the median value of cell densities. Thus, in some embodiments, the predetermined reference value thus allows discrimination between a poor and a good prognosis with respect to DFS and OS for a patient. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above. For example, according to this specific embodiment of a “cut-off” value, the outcome can be determined by comparing the density of CD8+ T cells with the range of values which are identified. In some embodiments, a cut-off value thus consists of a range of quantification values, e.g. centered on the quantification value for which the highest statistical significance value is found (e.g. generally the minimum p value which is found).

In some embodiments, the method of the present invention is particularly suitable for the treatment of cancer characterized by a high tumor infiltration of Treg cells.

As used herein, the term “regulatory T cells” or “Treg cells” refers to cells that suppress, inhibit or prevent T cells activity, in particular cytotoxic activity of T CD8+ cells. Regulatory T cells include i) thymus-derived Treg cells (tTreg, previously referred as “natural Treg cells”) and ii) peripherally-derived Treg cells (pTreg, previously referred as “induced Treg cells”). As used herein, tTregs have the following phenotype at rest CD4+CD25+FoxP3+. pTreg cells include, for example, Tr1 cells, TGF-β secreting Th3 cells, regulatory NKT cells, regulatory γδ T cells, regulatory CD8+ T cells, and double negative regulatory T cells. The term “Tr1 cells” as used herein refers to cells having the following phenotype at rest: CD4+CD25−CD127−, and the following phenotype when activated: CD4+CD25+CD127−. Tr1 cells, Type 1 T regulatory cells (Type 1 Treg) and IL-10 producing Treg are used herein with the same meaning. In one embodiment, Tr1 cells may be characterized, in part, by their unique cytokine profile: they produce IL-10, and IFN-gamma, but little or no IL-4 or IL-2. In one embodiment, Tr1 cells are also capable of producing IL-13 upon activation. The term “Th3 cells” as used herein refers to cells having the following phenotype CD4+FoxP3+ and capable of secreting high levels TGF-β upon activation, low amounts of IL-4 and IL-10 and no IFN-γ or IL-2. These cells are TGF-β derived. The term “regulatory NKT cells” as used herein refers to cells having the following phenotype at rest CD161+CD56+CD16+ and expressing a Vα24/Vβ11 TCR. The term “regulatory CD8+ T cells” as used herein refers to cells having the following phenotype at rest CD8+CD122+ and capable of secreting high levels of IL-10 upon activation. The term “double negative regulatory T cells” as used herein refers to cells having the following phenotype at rest TCRαβ+CD4−CD8−. The term “γδ T cells” as used herein refers to T lymphocytes that express the [gamma] [delta] heterodimer of the TCR. Unlike the [alpha] [beta] T lymphocytes, they recognize non-peptide antigens via a mechanism independent of presentation by MHC molecules. Two populations of γδ T cells may be described: the γδ T lymphocytes with the V γ9V δ2 receptor, which represent the majority population in peripheral blood and the γδ T lymphocytes with the V δ1 receptor, which represent the majority population in the mucosa and have only a very limited presence in peripheral blood. V γ9V δ2 T lymphocytes are known to be involved in the immune response against intracellular pathogens and hematological diseases.

As described for CD8+ T cells the tumor-inflitration of Treg cells is determined by any convention method in the art. In particular, said determination comprises quantifying the density of Treg cells T cells in a tumor sample obtained from the patient, in particular by immunohistochemistry (IHC). Accordingly, IHC methods described for determining the density of CD8+ T cells apply mutatis mutandis for measuring the density of Treg cells provided that binding partners (e.g. antibodies) specific for Tregs are used.

In some embodiments, the cell density of Treg cells is determined in the whole tumor tissue sample, is determined in the invasive margin or centre of the tumor tissue sample or is determined both in the centre and the invasive margin of the tumor tissue sample.

Accordingly a further object of the present invention relates to a method of treating cancer in a patient in need thereof comprising i) quantifying the density of Treg cells in a tumor tissue sample obtained from the patient ii) comparing the density quantified at step i) with a predetermined reference value and iii) administering to the patient a therapeutically effective combination of chemotherapy and immunotherapy with the caloric restriction mimetic when the density determined at step i) is higher than the predetermined reference value.

The methods described for determining the predetermined reference values for CD8+ T cells apply mutatis mutandis for Treg cells.

In some embodiments, the method of the present invention is particularly suitable for the treatment of cancer characterized by a low tumor infiltration of CD8+ T cells and a high tumor infiltration of Treg cells.

Accordingly a further object of the present invention relates to a method of treating cancer in a patient in need thereof comprising i) quantifying the density of Treg cells and CD8+ T cells in a tumor tissue sample obtained from the patient ii) comparing the densities quantified at step i) with their predetermined reference values and iii) administering to the patient a therapeutically effective combination of chemotherapy and immunotherapy with the caloric restriction mimetic when the density for Tregs quantified at step i) is higher than its corresponding predetermined reference value and the density quantified for CD8+ T cells quantified at step i) lower that its corresponding predetermined reference value.

In some embodiments, the cancer is a KRAS mutated cancer. As used herein, “KRAS” refers to v-Ki-ras2 Kirsten rat sarcoma viral oncogene homolog. KRAS is also known in the art as NS3, KRAS1, KRAS2, RASK2, KI-RAS, C-K-RAS, K-RAS2A, K-RAS2B, K-RAS4A and K-RAS4B. This gene, a Kirsten ras oncogene homolog from the mammalian ras gene family, encodes a protein that is a member of the small GTPase superfamily A single amino acid substitution can be responsible for an activating mutation. The transforming protein that results can be implicated in various malignancies, including lung cancer, colon cancer and pancreas cancer. KRAS mutations are well known in the art and are frequently found in neoplasms include those at exon 1 (codons 12 and 13) and exon 2 (codon 61) (e.g., the 34A, 34C, 34T, 35A, 35C, 35T or 38A mutations). Other examples of KRAS mutations include, but are not limited to, G12A, G12D, G12R, G12C, G12S, G12V and G13D. Somatic KRAS mutations are found at high rates in leukemias, colorectal cancer (Burmer et al. Proc. Natl. Acad. Sci. 1989 86: 2403-7), pancreatic cancer (Almoguera et al. Cell 1988 53: 549-54) and lung cancer (Tam et al. Clin. Cancer Res. 2006 12: 1647-53). Methods for identifying KRAS mutations are well known in the art and are commercially available (e.g. In Therascreen (Qiagen) assay, Taqman® Mutation Detection Assays powered by castPCR™ technology (Life Technologies)).

In some embodiments, the cancer is an autophagy competent cancer. As used herein the term “autophagy competent cancer” denotes a cancer wherein autophagy could occur. In some embodiments, an ATG5 or ATG7 deficiency is not detected. In the context of the invention, the term “ATG5 or ATG7 deficiency” denotes that the tumour cells of the subject or a part thereof have an ATG5 or ATG7 dysfunction, a low or a null expression of ATG5 or ATG7 gene. Said deficiency may typically result from a mutation in ATG5 or ATG7 gene so that the pre-ARNm is degraded through the NMD (non sense mediated decay) system. Said deficiency may also typically result from a mutation so that the protein is misfolded and degraded through the proteasome. Said deficiency may also result from a loss of function mutation leading to a dysfunction of the protein. Said deficiency may also result from an epigenetic control of gene expression (e.g. methylation) so that the gene is less expressed in the cells of the subject. Said deficiency may also result from a repression of the ATG5 or ATG7 gene induce by a particular signaling pathway. Said deficiency may also result from a mutation in a nucleotide sequence that control the expression of ATG5 or ATG7 gene. As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “chemotherapy” has its general meaning in the art and refers to the treatment that consists in administering to the patient a chemotherapeutic agent. In some embodiments, the chemotherapeutic agent is an immunogenic cell death (ICD) inducer, i.e. a pharmacological compounds that kills malignant cells in a way that they elicit an anticancer immune response.(10-19) Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluoromethylomithine (DMFO); retinoids such as retinoic acid; capecitabine; and pharmaceutically acceptable salts thereof. In one embodiment, the chemotherapeutic agents of the include pharmaceutically acceptable acids or derivatives of any of the above.

In some embodiments, the chemotherapeutic agent is selected from the group consisting of anthracyclines, oxaliplatin and taxanes. As used herein, the term “anthracycline” refers to a class of antineoplastic antibiotics having an anthracenedione (also termed anthraquinone or dioxoanthracene) structural unit. For example, the term “anthracycline” is specifically intended to individually include daunorubicin, doxorubicin, epirubicin, idarubicin, valrubicin, ditrisarubicins, mitoxantrone, etc. As used herein, the term “taxane” has its general meaning in the art and is used to identify a diterpene moiety that is only slightly soluble in water. Taxanes according to the invention include without limitation moieties isolated from the Pacific yew tree (Taxus brevifolia) as well as derivatives, analogs, metabolites and prodrugs, and other taxanes. Preferably, the taxane is selected from the group consisting of paclitaxel, docetaxel, derivatives, analogs, metabolites and prodrugs of paclitaxel or docetaxel, and salts, polymorphs and hydrates thereof. As used herein, the term “oxaliplatin” refers to [(1R,2R.)-cyclohexane-1,2-diamine](ethanedioato-O,O′)platinum(II) (1,2 Diamino-cyclohexane Platinum Oxalate, Chemical Abstracts Services Registry No. 63121-00-6).

The Applicant has evidenced the effectiveness of the present invention independently of the chemical or pharmacological nature of the chemotherapeutic agent.

In one embodiment, the chemotherapeutic agent is at least one agent selected from list A consisting of cyclophosphamide, dolastatin, pancratistatin, mechlorethamine, bleomycins, dactinomycin, daunorubicin, doxorubicin, morpholino-doxorubicin, cyanomorpholino-doxorubicin, 5-pyrrolino-doxorubicin, deoxy doxorubicin, epirubicin, idarubicin, 8-fluorouracil (5-FU), trimetrexate, epothilones, lonidamine, maytansine, mitoxantrone, PSK polysaccharide complex, verrucarin A, vindesine, cytosine arabinoside (“Ara-C”), paclitaxel, docetaxel, 9-thioguanine, cisplatin, oxaliplatin, carboplatin, vinblastine, platinum, ansamitocins, vincristine, vinorelbine, novantrone (mitoxantrone), daunomycin (=daunorubicin), irinotecan (e.g., CPT-1 1), retinoic acid, bortezomib, digitoxin, digoxin, patupilone, hypericin, cetuximab, septacidin, hedamycin, CDDP, mitomycin C, temozolomide, pemetrexed, camptothecin, bryostatin, spongistatin, chlorambucil, ifosfamide, mechlorethamine oxide hydrochloride, melphalan, trofosfamide, chlorozotocin, fotemustine, calicheamicin, enediyne antiobiotic chromophores, actinomycin, azaserine, hydroxyurea, mycophenolic acid, peplomycin, puromycin, streptonigrin, ubenimex/bestatin, methotrexate, thioguanine, carmofur, cytarabine, dideoxyuridine (“deoxyuridine”), aldophosphamide glycoside, amsacrine, diaziquone, lentinan, mitoguazone, pentostatin, pirarubicin, losoxantrone, rhizoxin, dacarbazine, thiotepa, neocarzinostatin chromophore, gemcitabine, etoposide (VP-16), teniposide, aminopterin, ibandronate, DFMO, germanium, panitumumab, erlotinib, mafosfamide, vemurafenib, busulfan, improsulfan, piposulfan, benzodopa, carboquone, meturedopa, uredopa, altretamine, triethylenemelamine, trietylenephosphoramide, triethiylenethiophosphoramide, trimethylolomelamine, bullatacin, bullatacinone, topotecan, callystatin, CC-1066, adozelesin, carzelesin, bizelesin, cryptophycin 1, cryptophycin 8, duocarmycin, KW-2190, CB1-TM2, eleutherobin, sarcodictyin, chlornaphazine, cholophosphamide, estramustine, novembichin, phenesterine, prednimustine, uracil mustard, carmustine, lomustine, nimustine, ranimnustine, dynemicin, clodronatc, esperamicin, aclacinomysins, authrarnycin, cactinomycin, carabicin, caminomycin, carzinophilin, chromomycins, detorubicin, 7-diazo-5-Oxo-L-norleucine, esorubicin, marcellomycin, nogalamycin, olivomycins, oligomycins, potfiromycin, quelamycin, rodorubicin, streptozocin, tubercidin, zinostatin, zorubicin, denopterin, pteropterin, fludarabine, 7-mercaptopurine, thiamiprine, ancitabine, azacitidine, 7-azauridine, doxifluridine, enocitabine, floxuridine, calusterone, dromostanolone propionate, epitiostanol, testolactone, anti-adrenals, aminoglutethimide, mitotane, trilostane, folinic acid, aceglatone, aminolevulinic acid, eniluracil, bestrabucil, bisantrene, edatraxate, demecolcine, elfornithine, elliptinium acetate, etoglucid, gallium nitrate, mopidanmol, nitraerine, phenamet, podophyllinic acid, 3-ethylhydrazide, procarbazine, razoxane, sizofuran, spirogermanium, tenuazonic acid, triaziquone, 3,2,2»-trichlorotriethylamine, T-2 toxin, roridin A, anguidine, urethane, mannomustine, mitobronitol, mitolactol, pipobroman, gacytosine, mercaptopurine, mepitiostane, edatrexate, xeloda (capecitabine), RFS 2001, capecitabine, defofamine, Abemaciclib, Abiraterone Acetate, Abraxane (Paclitaxel Albumin-stabilized Nanoparticle Formulation), ABVD, ABVE, ABVE-PC, AC, Acalabrutinib, AC-T, Actemra (Tocilizumab), Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ameluz (Aminolevulinic Acid), Amifostine, Aminolevulinic Acid, Anastrozole, Apalutamide, Aprepitant, Aranesp (Darbepoetin Alfa), Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Asparlas (Calaspargase Pegol-mknl), Atezolizumab, Avastin (Bevacizumab), Avelumab, Axicabtagene Ciloleucel, Axitinib, Azacitidine, Azedra (Iobenguane I 131), Bavencio (Avelumab), BEACOPP, Beleodaq (Belinostat), Belinostat, Bendamustine Hydrochloride, Bendeka (Bendamustine Hydrochloride), BEP, Besponsa (Inotuzumab Ozogamicin), Bevacizumab, Bexarotene, Bicalutamide, BiCNU (Carmustine), Binimetinib, Bleomycin, Blinatumomab, Blincyto (Blinatumomab), Bortezomib, Bosulif (Bosutinib), Bosutinib, Braftovi (Encorafenib), Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Calaspargase Pegol-mknl, Calquence (Acalabrutinib), Campath (Alemtuzumab), Camptosar (Irinotecan Hydrochloride), Capecitabine, CAPDX, Carac (Fluorouracil—Topical), Carboplatin, CARBOPLATIN-, Carfilzomib, Carmustine, Carmustine Implant, Casodex (Bicalutamide), CEM, Cemiplimab-rwlc, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clofarabine, Clolar (Clofarabine), CMF, Cobimetinib, Cometriq (Cabozantinib-S-Malate), Copanlisib Hydrochloride, COPDAC, Copiktra (Duvelisib), COPP, COPP-ABV, Cosmegen (Dactinomycin), Cotellic (Cobimetinib), Crizotinib, CVP, Cyclophosphamide, Cyramza (Ramucirumab), Cytarabine, Cytarabine Liposome, Cytosar-U (Cytarabine), Dabrafenib, Dacarbazine, Dacogen (Decitabine), Dacomitinib, Dactinomycin, Daratumumab, Darbepoetin Alfa, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), Durvalumab, Duvelisib, Efudex (Fluorouracil—Topical), Eligard (Leuprolide Acetate), Elitek (Rasburicase), Ellence (Epirubicin Hydrochloride), Elotuzumab, Eloxatin (Oxaliplatin), Eltrombopag Olamine, Elzonris (Tagraxofusp-erzs), Emapalumab-lzsg, Emend (Aprepitant), Empliciti (Elotuzumab), Enasidenib Mesylate, Encorafenib, Enzalutamide, Epirubicin Hydrochloride, EPOCH, Epoetin Alfa, Epogen (Epoetin Alfa), Erbitux (Cetuximab), Eribulin Mesylate, Erivedge (Vismodegib), Erleada (Apalutamide), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Firmagon (Degarelix), Fludarabine Phosphate, Fluoroplex (Fluorouracil—Topical), Fluorouracil Injection, Fluorouracil—Topical, Flutamide, FOLFIRI, FOLFIRI-BEVACIZUMAB, FOLFIRI-CETUXIMAB, FOLFIRINOX, FOLFOX, Folotyn (Pralatrexate), Fostamatinib Disodium, FU-LV, Fulvestrant, Fusilev (Leucovorin Calcium), Gamifant (Emapalumab-lzsg), Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gilteritinib Fumarate, Gleevec (Imatinib Mesylate), Gliadel Wafer (Carmustine Implant), Glucarpidase, Goserelin Acetate, Granisetron, Granisetron Hydrochloride, Granix (Filgrastim), Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idarubicin Hydrochloride, Idelalisib, Idhifa (Enasidenib Mesylate), Ifex (Ifosfamide), Ifosfamide, IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iobenguane I 131, Ipilimumab, Iressa (Gefitinib), Irinotecan Hydrochloride, Irinotecan Hydrochloride Liposome, Istodax (Romidepsin), Ivosidenib, Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Larotrectinib Sulfate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Levulan Kerastik (Aminolevulinic Acid), Libtayo (Cemiplimab-rwlc), LipoDox (Doxorubicin Hydrochloride Liposome), Lomustine, Lonsurf (Trifluridine and Tipiracil Hydrochloride), Lorbrena (Lorlatinib), Lorlatinib, Lumoxiti (Moxetumomab Pasudotox-tdfk), Lupron (Leuprolide Acetate), Lupron Depot (Leuprolide Acetate), Lutathera (Lutetium Lu 177-Dotatate), Lutetium (Lu 177-Dotatate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Mektovi (Binimetinib), Melphalan, Melphalan Hydrochloride, Mercaptopurine, Mesna, Mesnex (Mesna), Methotrexate, Methylnaltrexone Bromide, Midostaurin, Mitomycin C, Mitoxantrone Hydrochloride, Mogamulizumab-kpkc, Moxetumomab Pasudotox-tdfk, Mozobil (Plerixafor), Mustargen (Mechlorethamine Hydrochloride), MVAC, Myleran (Busulfan), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation (nab-paclitaxel), PAD, Palbociclib, Palifermin, Palonosetron Hydrochloride, Palonosetron Hydrochloride and Netupitant, Pamidronate Disodium, Panitumumab, Panobinostat, Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Plerixafor, Pomalidomide, Pomalyst (Pomalidomide), Ponatinib Hydrochloride, Portrazza (Necitumumab), Poteligeo (Mogamulizumab-kpkc), Pralatrexate, Prednisone, Procarbazine Hydrochloride, Procrit (Epoetin Alfa), Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, Ravulizumab-cwvz, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Retacrit (Epoetin Alfa), Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sancuso (Granisetron), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sustol (Granisetron), Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagraxofusp-erzs, Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Tavalisse (Fostamatinib Disodium), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tibsovo (Ivosidenib), Tisagenlecleucel, Tocilizumab, Tolak (Fluorouracil—Topical), Topotecan Hydrochloride, Toremifene, Torisel (Temsirolimus), Totect (Dexrazoxane Hydrochloride), TPF, Trabectedin, Trametinib, Trastuzumab, Treanda (Bendamustine Hydrochloride), Trexall (Methotrexate), Trifluridine and Tipiracil Hydrochloride, Trisenox (Arsenic Trioxide), Tykerb (Lapatinib Ditosylate), Ultomiris (Ravulizumab-cwvz), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VeIP, Velcade (Bortezomib), Vemurafenib, Venclexta (Venetoclax), Venetoclax, Verzenio (Abemaciclib), Vidaza (Azacitidine), Vinblastine Sulfate, Vincristine Sulfate, Vincristine Sulfate Liposome, Vinorelbine Tartrate, VIP, Vismodegib, Vistogard (Uridine Triacetate), Vitrakvi (Larotrectinib Sulfate), Vizimpro (Dacomitinib), Voraxaze (Glucarpidase), Vorinostat, Votrient (Pazopanib Hydrochloride), Vyxeos (Daunorubicin Hydrochloride and Cytarabine Liposome), Xalkori (Crizotinib), Xeloda (Capecitabine), XELIRI, XELOX, Xgeva (Denosumab), Xofigo (Radium 223 Dichloride), Xospata (Gilteritinib Fumarate), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), Zytiga (Abiraterone Acetate), cyclophosphamide, dolastatin, pancratistatin, mechlorethamine, bleomycins, dactinomycin, daunorubicin, doxorubicin, morpholino-doxorubicin, cyanomorpholino-doxorubicin, 2-pyrrolino-doxorubicin, deoxy doxorubicin, epirubicin, idarubicin, 5-fluorouracil (5-FU), trimetrexate, epothilones, lonidamine, maytansine, mitoxantrone, PSK polysaccharide complex, verrucarin A, vindesine, cytosine arabinoside («Ara-C”), paclitaxel, docetaxel, nab-paclitaxel, 6-thioguanine, cisplatin, oxaliplatin, carboplatin, vinblastine, platinum, ansamitocins, vincristine, vinorelbine, novantrone (mitoxantrone), daunomycin (=daunorubicin), irinotecan (e.g., CPT-1 1), retinoic acid, bortezomib, digitoxin, digoxin, patupilone, hypericin, cetuximab, septacidin, hedamycin, CDDP, mitomycin C, temozolomide and pemetrexed.

In one embodiment, the chemotherapeutic agent is at least one agent selected from list B consisting of cyclophosphamide, dolastatin, pancratistatin, mechlorethamine, bleomycins, dactinomycin, daunorubicin, doxorubicin, morpholino-doxorubicin, cyanomorpholino-doxorubicin, 5-pyrrolino-doxorubicin, deoxy doxorubicin, epirubicin, idarubicin, 8-fluorouracil (5-FU), trimetrexate, epothilones, lonidamine, maytansine, mitoxantrone, PSK polysaccharide complex, verrucarin A, vindesine, cytosine arabinoside («Ara-C”), paclitaxel, docetaxel, 9-thioguanine, cisplatin, oxaliplatin, carboplatin, vinblastine, platinum, ansamitocins, vincristine, vinorelbine, novantrone (mitoxantrone), daunomycin (=daunorubicin), irinotecan (e.g., CPT-1 1), retinoic acid, bortezomib, digitoxin, digoxin, patupilone, hypericin, cetuximab, septacidin, hedamycin, CDDP, mitomycin C, temozolomide, pemetrexed, camptothecin, bryostatin, spongistatin, chlorambucil, ifosfamide, mechlorethamine oxide hydrochloride, melphalan, trofosfamide, chlorozotocin, fotemustine, calicheamicin, enediyne antiobiotic chromophores, actinomycin, azaserine, hydroxyurea, mycophenolic acid, peplomycin, puromycin, streptonigrin, ubenimex/bestatin, methotrexate, thioguanine, carmofur, cytarabine, dideoxyuridine (“deoxyuridine”), aldophosphamide glycoside, amsacrine, diaziquone, lentinan, mitoguazone, pentostatin, pirarubicin, losoxantrone, rhizoxin, dacarbazine, thiotepa, neocarzinostatin chromophore, gemcitabine, etoposide (VP-16), teniposide, aminopterin, ibandronate, DFMO, germanium, panitumumab, erlotinib, mafosfamide, vemurafenib, busulfan, improsulfan, piposulfan, benzodopa, carboquone, meturedopa, uredopa, altretamine, triethylenemelamine, trietylenephosphoramide, triethiylenethiophosphoramide, trimethylolomelamine, bullatacin, bullatacinone, topotecan, callystatin, CC-1066, adozelesin, carzelesin, bizelesin, cryptophycin 1, cryptophycin 8, duocarmycin, KW-2190, CB1-TM2, eleutherobin, sarcodictyin, chlornaphazine, cholophosphamide, estramustine, novembichin, phenesterine, prednimustine, uracil mustard, carmustine, lomustine, nimustine, ranimnustine, dynemicin, clodronatc, esperamicin, aclacinomysins, authrarnycin, cactinomycin, carabicin, caminomycin, carzinophilin, chromomycins, detorubicin, 7-diazo-5-Oxo-L-norleucine, esorubicin, marcellomycin, nogalamycin, olivomycins, oligomycins, potfiromycin, quelamycin, rodorubicin, streptozocin, tubercidin, zinostatin, zorubicin, denopterin, pteropterin, fludarabine, 7-mercaptopurine, thiamiprine, ancitabine, azacitidine, 7-azauridine, doxifluridine, enocitabine, floxuridine, calusterone, dromostanolone propionate, epitiostanol, testolactone, anti-adrenals, aminoglutethimide, mitotane, trilostane, folinic acid, aceglatone, aminolevulinic acid, eniluracil, bestrabucil, bisantrene, edatraxate, demecolcine, elfornithine, elliptinium acetate, etoglucid, gallium nitrate, mopidanmol, nitraerine, phenamet, podophyllinic acid, 3-ethylhydrazide, procarbazine, razoxane, sizofuran, spirogermanium, tenuazonic acid, triaziquone, 3,2,2-trichlorotriethylamine, T-2 toxin, roridin A, anguidine, urethane, mannomustine, mitobronitol, mitolactol, pipobroman, gacytosine, mercaptopurine, mepitiostane, edatrexate, xeloda (capecitabine), RFS 2001, capecitabine and defofamine

In one embodiment, the chemotherapeutic agent is at least one agent selected from list C consisting of cyclophosphamide, dolastatin, pancratistatin, mechlorethamine, bleomycins, dactinomycin, daunorubicin, doxorubicin, morpholino-doxorubicin, cyanomorpholino-doxorubicin, 5-pyrrolino-doxorubicin, deoxy doxorubicin, epirubicin, idarubicin, 8-fluorouracil (5-FU), trimetrexate, epothilones, lonidamine, maytansine, mitoxantrone, PSK polysaccharide complex, verrucarin A, vindesine, cytosine arabinoside (“Ara-C”), paclitaxel, docetaxel, nab-paclitaxel, 9-thioguanine, cisplatin, oxaliplatin, carboplatin, vinblastine, platinum, ansamitocins, vincristine, vinorelbine, novantrone (mitoxantrone), daunomycin (=daunorubicin), irinotecan (e.g, CPT-1 1), retinoic acid, bortezomib, digitoxin, digoxin, patupilone, hypericin, cetuximab, septacidin, hedamycin, CDDP, mitomycin C, temozolomide, pemetrexed, camptothecin, bryostatin, spongistatin, chlorambucil, ifosfamide, mechlorethamine oxide hydrochloride, melphalan, trofosfamide, chlorozotocin, fotemustine, calicheamicin, enediyne antiobiotic chromophores, actinomycin, azaserine, hydroxyurea, mycophenolic acid, peplomycin, puromycin, streptonigrin, ubenimex/bestatin, methotrexate, thioguanine, carmofur, cytarabine, dideoxyuridine (“deoxyuridine”), aldophosphamide glycoside, amsacrine, diaziquone, lentinan, mitoguazone, pentostatin, pirarubicin, losoxantrone, rhizoxin, dacarbazine, thiotepa, neocarzinostatin chromophore, gemcitabine, etoposide (VP-16), teniposide, aminopterin, ibandronate, DFMO, germanium, panitumumab, erlotinib, mafosfamide and vemurafenib.

In one embodiment, the chemotherapeutic agent is at least one agent selected from list D consisting of cyclosphosphamide, dolastatin, pancratistatin, mechlorethamine, bleomycins, dactinomycin, daunorubicin, doxorubicin, morpholino-doxorubicin, cyanomorpholino-doxorubicin, 2-pyrrolino-doxorubicin, deoxy doxorubicin, epirubicin, idarubicin, 5-fluorouracil (5-FU), trimetrexate, epothilones, lonidamine, maytansine, mitoxantrone, PSK polysaccharide complex, verrucarin A, vindesine, cytosine arabinoside (“Ara-C”), paclitaxel, docetaxel, 6-thioguanine, cisplatin, oxaliplatin, carboplatin, vinblastine, platinum, ansamitocins, vincristine, vinorelbine, novantrone (mitoxantrone), daunomycin (=daunorubicin), irinotecan (e.g., CPT-1 1), retinoic acid, bortezomib, digitoxin, digoxin, patupilone, hypericin, cetuximab, septacidin, hedamycin, CDDP, mitomycin C, temozolomide and pemetrexed.

In one typical embodiment, the at least one chemotherapeutic agent is selected from list E consisting of:
platinum coordination complexes selected from cisplatin, oxaliplatin and carboplatin;
taxanes selected from paclitaxel, nab-paclitaxel, docetaxel and taxotere;
vinca alkaloids selected from vindesine vinblastine vincristine and vinorelbine; and
anthracyclines selected from mitoxantrone daunorubicin, doxorubicin, epirubicin, idarubicin, valrubicin and ditrisarubicin;
gemcitabine
pemetrexed
mixtures thereof and pharmaceutically acceptable salts thereof.

In one typical embodiment, the at least one chemotherapeutic agent is selected from list F consisting of:
platinum coordination complexes selected from cisplatin, oxaliplatin and carboplatin;
taxanes selected from paclitaxel, nab-paclitaxel, docetaxel and taxotere;
anthracyclines selected from mitoxantrone daunorubicin, doxorubicin, epirubicin, idarubicin, valrubicin and ditrisarubicin; and
mixtures thereof and pharmaceutically acceptable salts thereof.

In one typical embodiment, the at least one chemotherapeutic agent is selected from list G consisting of:
cisplatin, oxaliplatin and carboplatin; or a simultaneous or sequential administration of carboplatin and pemetrexed; or a simultaneous or sequential administration of oxaliplatin and 5-FU
taxanes selected from paclitaxel, nab-paclitaxel, docetaxel and taxotere;
gemcitabine
5-FU
pemetrexed
anthracyclines selected from mitoxantrone daunorubicin, doxorubicin, epirubicin, idarubicin, valrubicin and ditrisarubicin; and mixtures thereof and pharmaceutically acceptable salts thereof.

In one typical embodiment, the at least one chemotherapeutic agent is selected from list H consisting of:
cisplatin, oxaliplatin and carboplatin; or a simultaneous or sequential administration of carboplatin and pemetrexed; or a simultaneous or sequential administration of oxaliplatin and 5-FU
taxanes selected from paclitaxel, nab-paclitaxel, docetaxel and taxotere;
gemcitabine
pemetrexed
mitoxantrone; and
mixtures thereof and pharmaceutically acceptable salts thereof.

In one typical embodiment, the at least one chemotherapeutic agent is oxaliplatin. In one typical embodiment, the at least one chemotherapeutic carboplatin. In one typical embodiment, the at least one chemotherapeutic agent is a simultaneous or sequential administration of carboplatin and pemetrexed. In one typical embodiment, the at least one chemotherapeutic agent is a simultaneous or sequential administration of oxaliplatin and 5-FU. In one typical embodiment, the at least one chemotherapeutic agent is gemcitabine. In one typical embodiment, the at least one chemotherapeutic agent is pemetrexed. In one typical embodiment, the at least one chemotherapeutic agent is mitoxantrone;

In one particular embodiment, the method according to the invention further comprises the application of radiotherapy, prior or posterior to the administration of the composition comprising at least one CRM as hereinafter described.

In one particular embodiment, the method according to the invention further comprises the application of radiotherapy, prior or posterior to the administration of the composition comprising at least one CRM as hereinafter described.

As used herein, the term “immunotherapy” has its general meaning in the art and refers to the treatment that consists in administering an immunogenic agent i.e. an agent capable of inducing, enhancing, suppressing or otherwise modifying an immune response.

In some embodiments, the immunotherapy consists in administering the patient with at least one immune checkpoint inhibitor. As used herein, the term “immune checkpoint inhibitor” has its general meaning in the art and refers to any compound inhibiting the function of an immune inhibitory checkpoint protein. As used herein the term “immune checkpoint protein” has its general meaning in the art and refers to a molecule that is expressed by T cells in that either turn up a signal (stimulatory checkpoint molecules) or turn down a signal (inhibitory checkpoint molecules) Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of inhibitory checkpoint molecules include A2AR, B7-H3, B7-H4, BTLA, CTLA-4, CD277, IDO, KIR, PD-1, LAG-3, TIM-3 and VISTA. Inhibition includes reduction of function, partial and full blockade. Preferred immune checkpoint inhibitors are antibodies that specifically recognize immune checkpoint proteins. A number of immune checkpoint inhibitors are known and in analogy of these known immune checkpoint protein inhibitors, alternative immune checkpoint inhibitors may be developed in the (near) future. The immune checkpoint inhibitors include peptides, antibodies, nucleic acid molecules and small molecules. Examples of immune checkpoint inhibitor includes PD-1 antagonist, PD-L1 antagonist, PD-L2 antagonist CTLA-4 antagonist, VISTA antagonist, TIM-3 antagonist, LAG-3 antagonist, IDO antagonist, KIR2D antagonist, A2AR antagonist, B7-H3 antagonist, B7-H4 antagonist, and BTLA antagonist.

In one embodiment, the at least one immune checkpoint inhibitor is selected from list I consisting of: anti PD1 agents, anti PDL1 agents, anti CTLA4 agents, PD-L2 antagonists, VISTA antagonists, TIM-3 antagonists, LAG-3 antagonists, IDO antagonists, KIR2D antagonists, A2AR antagonists, B7-H3 antagonists, B7-H3 antagonists, B7-H4 antagonists, BTLA antagonists, Vx-001, a therapeutic vaccine based on optimized cryptic peptides (Vaxon biotech), Dendritic cell therapy, CAR-T cell therapy, Nivolumab, Pembrolizumab, Pidilizumab, AMP-224, Atezolimumab, Avelumab, CA-170, BMS-936559, Durvalumab, MCLA-145, SP142, STI-A1011, STIA1012, STI-A1010, STI-A1014, A110, KY1003, Ipilimumab, Tremelimumab, Dendritic cell therapy, CAR-T cell therapy, IMP320, MGA270, anti-TIM2, 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 4-methyl tryptophan, 6-methyl-tryptophan, 5-methoxy-tryptophan, 4-hydroxy-tryptophan, 19 indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole, IL-1α, IL-1β, IL-1Ra (antagonist), IL-2, IL-3, IL-4, IL-5, IL-6, IL-7, IL-8, IL-9, IL-10, IL-11, IL-12 (p35/p40), IL-13, IL-14, IL-15, IL-16, IL-17A, IL-17B,C,D, IL-17F, IL-18, IL-19, IL-20, IL-21, IL-22, IL-23, (P19+), IL-24, IL-25, (IL-17E), IL-26, IL-27, P28+EB13), IL-28A/B/IL-29, IL-30 (p28 subunit of IL-27) antagonists, IL-1α, IL-1β antagonists, IL-1Ra antagonists, IL-2 antagonists, IL-3 antagonists, IL-4 antagonists, IL-5 antagonists, IL-6 antagonists, IL-7 antagonists, IL-8 antagonists, IL-9 antagonists, IL-10 antagonists, IL-11 antagonists, IL-12 (p35/p40) antagonists, IL-13 antagonists, IL-14 antagonists, IL-15 antagonists, IL-16 antagonists, IL-17A antagonists, IL-17B,C,D antagonists, IL-17F antagonists, IL-18 antagonists, IL-19 antagonists, IL-20 antagonists, IL-21 antagonists, IL-22 antagonists, IL-23 antagonists, (P19+) antagonists, IL-24 antagonists, IL-25 antagonists, (IL-17E) antagonists, IL-26 antagonists, IL-27 antagonists, P28+EB13 antagonists, IL-28A/B/IL-29, IL-30 (p28 subunit of IL-27), Ipilimumab, Avelumab, Atezolizumab, Anti-GD2 antibodies, Anti-CD47 therapy, Adoptive T-cell therapy, CISH inhibitor, Oncolytic virus, Interferon type 1, Interferon type 2, Interferon type 3, Cryoimmunotherapy/cryoablation, Photoimmunotherapy and cancer vaccines.

In the context of the present invention, applying Cryoimmunotherapy or cryoablation Photoimmunotherapy or cancer vaccines may be interpreted as adiministrating the effects of a check-point inhibitor.

Typically, the immune therapy is selected from PD-1 antagonists, PD-L1 antagonists, CTLA-4 antagonists and mixtures thereof.

Typically, the immune therapy is selected from:
PD-1 antagonists such as Nivolumab, Pembrolizumab and Pidilizumab,
PD-L1 antagonists such as Avelumab, BMS-936559, CA-170, Durvalumab, MCLA-145, SP142, STI-A1011, STIA1012, STI-A1010, STI-A1014, A110, KY1003 and Atezolimumab,
CTLA-4 antagonists such as Tremelimumab and Ipilimumab, and
mixtures thereof.

In one typical embodiment, the at least one immune checkpoint inhibitor is selected from list J consisting of: Nivolumab, Pembrolizumab, Pidilizumab, AMP-224, Atezolimumab, Avelumab, CA-170, BMS-936559, Durvalumab, MCLA-145, SP142, STI-A1011, STIA1012, STI-A1010, STI-A1014, A110, KY1003, Ipilimumab, Tremelimumab, Dendritic cell therapy, CAR-T cell therapy, IMP320, MGA270, anti-TIM2, 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 4-methyl tryptophan, 6-methyl-tryptophan, 5-methoxy-tryptophan, 4-hydroxy-tryptophan, 19 indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole, Vx-001, a therapeutic vaccine based on optimized cryptic peptides (Vaxon biotech), Anti-GD2 antibodies, Anti-CD47 therapy, Adoptive T-cell therapy, CISH inhibitor, Oncolytic virus, Interferon type 1, Interferon type 2, Interferon type 3, Cryoimmunotherapy/cryoablation, Photoimmunotherapy, cancer vaccines, IL2, IL6 antagonist, IL4 antagonist, IL10 antagonists and IL10 agonists.

In one typical embodiment, the at least one immune checkpoint inhibitor is selected from list K consisting of: Nivolumab, Pembrolizumab, Pidilizumab, AMP-224, Atezolimumab, Avelumab, CA-170, BMS-936559, Durvalumab, MCLA-145, SP142, STI-A1011, STIA1012, STI-A1010, STI-A1014, A110, KY1003, Ipilimumab, Tremelimumab, Dendritic cell therapy, CAR-T cell therapy, IMP320, MGA270, anti-TIM2, 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 4-methyl tryptophan, 6-methyl-tryptophan, 5-methoxy-tryptophan, 4-hydroxy-tryptophan, 19 indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole and Vx-001, a therapeutic vaccine based on optimized cryptic peptides (Vaxon biotech).

In one typical embodiment, the at least one immune checkpoint inhibitor is selected from list L consisting of: Nivolumab, Pembrolizumab, Pidilizumab, AMP-224, Atezolimumab, Avelumab, CA-170, BMS-936559, Durvalumab, MCLA-145, SP142, STI-A1011, STIA1012, STI-A1010, STI-A1014, A110, KY1003, Ipilimumab and Tremelimumab.

In one typical embodiment, the at least one immune checkpoint inhibitor is selected from list L consisting of: Nivolumab, Pembrolizumab, Pidilizumab, AMP-224, Atezolimumab, Avelumab, CA-170, BMS-936559, Durvalumab, MCLA-145, SP142, STI-A1011, STIA1012, STI-A1010, STI-A1014, A110, KY1003, Ipilimumab and Tremelimumab.

In one typical embodiment, the at least one immune checkpoint inhibitor is selected from list M consisting of: Nivolumab, Pembrolizumab, Pidilizumab, Atezolimumab, Avelumab, Durvalumab, Ipilimumab and Tremelimumab.

In one embodiment, the at least one immune checkpoint inhibitor is Nivolumab. In one embodiment, the at least one immune checkpoint inhibitor is Pembrolizumab. In one embodiment, the at least one immune checkpoint inhibitor is Pidilizumab. In one embodiment, the at least one immune checkpoint inhibitor is Atezolimumab. In one embodiment, the at least one immune checkpoint inhibitor is Avelumab. In one embodiment, the at least one immune checkpoint inhibitor is Durvalumab. In one embodiment, the at least one immune checkpoint inhibitor is Ipilimumab. In one embodiment, the at least one immune checkpoint inhibitor is Tremelimumab.

In some embodiments, PD-1 (Programmed Death-1) axis antagonists include PD-1 antagonist (for example anti-PD-1 antibody), PD-L1 (Programmed Death Ligand-1) antagonist (for example anti-PD-L1 antibody) and PD-L2 (Programmed Death Ligand-2) antagonist (for example anti-PD-L2 antibody). In some embodiments, the anti-PD-1 antibody is selected from the group consisting of MDX-1106 (also known as Nivolumab, MDX-1106-04, ONO-4538, BMS-936558, and Opdivo®), Merck 3475 (also known as Pembrolizumab, MK-3475, Lambrolizumab, Keytruda®, and SCH-900475), and CT-011 (also known as Pidilizumab, hBAT, and hBAT-1). In some embodiments, the PD-1 binding antagonist is AMP-224 (also known as B7-DCIg). In some embodiments, the anti-PD-L1 antibody is selected from the group consisting of YW243.55.570, MPDL3280A, MDX-1105, and MEDI4736. MDX-1105, also known as BMS-936559, is an anti-PD-L1 antibody described in WO2007/005874. Antibody YW243.55. S70 is an anti-PD-L1 described in WO 2010/077634 Al. MEDI4736 is an anti-PD-L1 antibody described in WO2011/066389 and US2013/034559. MDX-1106, also known as MDX-1106-04, ONO-4538 or BMS-936558, is an anti-PD-1 antibody described in U.S. Pat. No. 8,008,449 and WO2006/121168. Merck 3745, also known as MK-3475 or SCH-900475, is an anti-PD-1 antibody described in U.S. Pat. No. 8,345,509 and WO2009/114335. CT-011 (Pidizilumab), also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342. Atezolimumab is an anti-PD-L1 antibody described in U.S. Pat. No. 8,217,149. Avelumab is an anti-PD-L1 antibody described in US 20140341917. CA-170 is a PD-1 antagonist described in WO2015033301 & WO2015033299. Other anti-PD-1 antibodies are disclosed in U.S. Pat. No. 8,609,089, US 2010028330, and/or US 20120114649. In some embodiments, the PD-1 inhibitor is an anti-PD-1 antibody chosen from Nivolumab, Pembrolizumab or Pidilizumab. In some embodiments, PD-L1 antagonist is selected from the group comprising of Avelumab, BMS-936559, CA-170, Durvalumab, MCLA-145, SP142, STI-A1011, STIA1012, STI-A1010, STI-A1014, A110, KY1003 and Atezolimumab and the preferred one is Avelumab, Durvalumab or Atezolimumab.

In some embodiments, CTLA-4 (Cytotoxic T-Lymphocyte Antigen-4) antagonists are selected from the group consisting of anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (Ipilimumab), Tremelimumab, anti-CD28 antibodies, anti-CTLA-4 adnectins, anti-CTLA-4 domain antibodies, single chain anti-CTLA-4 fragments, heavy chain anti-CTLA-4 fragments, light chain anti-CTLA-4 fragments, inhibitors of CTLA-4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B. Additional CTLA-4 antibodies are described in U.S. Pat. Nos. 5,811,097; 5,855,887; 6,051,227; and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17): 10067-10071 (1998); Camacho et al., J. Clin: Oncology, 22(145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281. A preferred clinical CTLA-4 antibody is human monoclonal antibody (also referred to as MDX-010 and Ipilimumab with CAS No. 477202-00-9 and available from Medarex, Inc., Bloomsbury, N.J.) is disclosed in WO 01/14424. With regard to CTLA-4 antagonist (antibodies), these are known and include Tremelimumab (CP-675,206) and Ipilimumab.

In some embodiments, the immunotherapy consists in administering to the patient a combination of a CTLA-4 antagonist and a PD-1 antagonist.

Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin.

Cancer Res. July 15 (18) 3834). Also included are TIM-3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94). As used herein, the term “TIM-3” has its general meaning in the art and refers to T cell immunoglobulin and mucin domain-containing molecule 3. The natural ligand of TIM-3 is galectin 9 (Ga19). Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signaling pathway and/or block the binding of TIM-3 to galectin-9. Antibodies having specificity for TIM-3 are well known in the art and typically those described in WO2011155607, WO2013006490 and WO2010117057.

In some embodiments, the immune checkpoint inhibitor is an IDO inhibitor. Examples of IDO inhibitors are described in WO 2014150677. Examples of IDO inhibitors include without limitation 1-methyl-tryptophan (IMT), (3-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. Preferably the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

As used herein, the term “caloric restriction mimetic” or “CRM” refers to any agent that mimics the biochemical and physiological consequences of caloric restriction and fasting. As used herein, the term “agent” refers to an entity capable of having a desired biological effect on a subject or cell. Examples of agents include small molecules (e.g., drugs), antibodies, peptides, proteins (e.g., cytokines, hormones, soluble receptors and nonspecific-proteins), oligonucleotides (e.g., peptide-coding DNA and RNA, double-stranded RNA and antisense RNA) and peptidomimetics.

In one embodiment CRMs are selected from inhibitors of ATP-citrate lyase, starvation, inhibitors of mitochondrial pyruvate carrier complex, inhibitors of CTP2 and inhibitors of mitochondrial citrate carrier (CIC).

In one embodiment, the at least one CRM is selected from hydroxy-citrate, lipoic acid,

EP300 acetyltransferase inhibitor, aspirin, salicylate, spermidine, anacardic acid, resveratrol, dicholoroacetate, quercetin, isoquercetin, valery salicylate, salsalate, saligenin, anacardic acid, balsalazide, 5-aminosalicylic acid, 4-aminosalicylic acid, alpha-cyanocinnamate derivative UK5099, perhexiline (PHX), benzenetricarboxylate (BTC), (R,S)—S-(3,4-dicarboxy-3-hydroxy-3methyl-butyl)-CoA, S-carboxymethyl-CoA, SB-204990, B M S-303141, epigallocatechine gallate, C646, ACCS2 inhibitor, SRT1721, ketoisocaproic acid, dimethyl-a-ketoglutarate, butyrate, 3-Methyladenine, Chloroquine, Bafilomycin A, oxaloacetate, metformin, rapamycin, glucosamine, N-acetyl-glucosamine, PPAR-gamma (Peroxisome proliferator-activated receptor gamma inhibitors) such as rosiglitazone, flavanols such as fisetin, Dipeptidyl peptidase 4 (DPP-4) inhibitors such as berberine, Sitagliptin, Vildagliptin, Saxagliptin, Linagliptin, Gemigliptin, Teneligliptin, Alogliptin, Trelagliptin, Omarigliptin, Evogliptin, Gosogliptin, Dutogliptin, 4-Phenylbutyrate and Gymnema sylvestre glycosides such as gymnemoside, pharmaceutically acceptable salts thereof and mixtures thereof.

In one embodiment, the at least one CRM is selected from hydroxy-citrate, lipoic acid, EP300 acetyltransferase inhibitor, spermidine, anacardic acid, resveratrol, dicholoroacetate, quercetin, isoquercetin, alpha-cyanocinnamate derivative UK5100, perhexiline (PHX), benzenetricarboxylate (BTC), (R,S)—S-(3,4-dicarboxy-3-hydroxy-3methyl-butyl)-CoA, S-carboxymethyl-CoA, SB-204991, B M S-303142, epigallocatechine gallate, C647, ACCS2 inhibitor, SRT1720, ketoisocaproic acid, dimethul-a-ketoglutarate, butyrate, 3-Methyladenine, Chloroquine, Bafilomycin A, oxaloacetate, metformin, rapamycin, glucosamine, N-acetyl-glucosamine, PPAR-gamma inhibitors (Peroxisome proliferator-activated receptor gamma inhibitors) such as rosiglitazone, flavanols such as fisetin, Dipeptidyl peptidase 4 (DPP-4) inhibitors such as berberine, Sitagliptin, Vildagliptin, Saxagliptin, Linagliptin, Gemigliptin, Teneligliptin, Alogliptin, Trelagliptin, Omarigliptin, Evogliptin, Gosogliptin, Dutogliptin, 4-Phenylbutyrate and Gymnema sylvestre glycosides such as gymnemoside, pharmaceutically acceptable salts thereof and mixtures thereof.

In one embodiment, the at least one CRM is selected from hydroxy-citrate, lipoic acid, EP300 acetyltransferase inhibitor, spermidine, anacardic acid, resveratrol, dicholoroacetate, quercetin, isoquercetin, alpha-cyanocinnamate derivative UK5100, perhexiline (PHX), benzenetricarboxylate (BTC), (R,S)—S-(3,4-dicarboxy-3-hydroxy-3methyl-butyl)-CoA, S-carboxymethyl-CoA, SB-204991, B M S-303142, epigallocatechine gallate, C647, ketoisocaproic acid, dimethul-a-ketoglutarate, butyrate, 3-Methyladenine, oxaloacetate, glucosamine, N-acetyl-glucosamine berberine gymnemoside, pharmaceutically acceptable salts thereof and mixtures thereof.

In one embodiment, the at least one CRM is selected from list N consisting of hydroxy-citrate, lipoic acid, EP300 acetyltransferase inhibitor spermidine, anacardic acid, resveratrol, dicholoroacetate, quercetin, isoquercetin, balsalazide, 5-aminosalicylic acid, 4-aminosalicylic acid, alpha-cyanocinnamate derivative UK5100, perhexiline (PHX), benzenetricarboxylate (BTC), (R,S)—S-(3,4-dicarboxy-3-hydroxy-3methyl-butyl)-CoA, S-carboxymethyl-CoA, SB-204990, B M S-303142, epigallocatechine gallate, C646, ACCS2 inhibitor, SRT1720, ketoisocaproic acid, dimethy-a-ketoglutarate, butyrate, 3-Methyladenine, Chloroquine, and BafilomycinA.

In one embodiment, the at least one CRM is not selected from salicylates.

In one embodiment, the at least one CRM is selected from list O consisting of hydroxy-citrate, lipoic acid, EP300 acetyltransferase inhibitor, spermidine, anacardic acid, resveratrol, dicholoroacetate, quercetin, isoquercetin, alpha-cyanocinnamate derivative UK5100, perhexiline (PHX), benzenetricarboxylate (BTC), (R,S)—S-(3,4-dicarboxy-3-hydroxy-3methyl-butyl)-CoA, S-carboxymethyl-CoA, SB-204990, B M S-303142, epigallocatechine gallate, C646, ACCS2 inhibitor, SRT1720, ketoisocaproic acid, dimethyl-a-ketoglutarate, butyrate, 3-Methyladenine, Chloroquine, and BafilomycinA.

In one embodiment, the at least one CRM is selected from list P consisting of hydroxy-citrate, lipoic acid, EP300 acetyltransferase inhibitor, spermidine, anacardic acid, resveratrol, dicholoroacetate, quercetin and isoquercetin.

In one embodiment the at least one CRM is selected from list Q consisting of hydroxycitrate, lipoic acid, spermidine; resveratrol, pharmaceutically acceptable salts thereof and mixtures thereof.

In one embodiment the at least one CRM is selected from list R consisting of hydroxycitrate, lipoic acid, pharmaceutically acceptable salts thereof and mixtures thereof.

In one embodiment CRMs are selected from hydroxycitrate, lipoic acid, spermidine, pharmaceutically acceptable salts thereof and mixtures thereof.

In one embodiment CRMs are selected from hydroxycitrate, lipoic acid, pharmaceutically acceptable salts thereof and mixtures thereof. In one particular embodiment, the CRM is hydroxycitrate. In one particular embodiment, the CRM is an association of hydroxycitrate with lipoic acid.

In one particular embodiment, CRMs stimulate autophagy by favoring the deacetylation of cellular proteins, mostly in the cyotoplasm of the cell. As used herein, the term “autophagy” refers to macroautophagy, unless stated otherwise, is the catabolic process involving the degradation of a cell's own components; such as, long lived proteins, protein aggregates, cellular organelles, cell membranes, organelle membranes, and other cellular components. The mechanism of autophagy may include: (i) the formation of a membrane around a targeted region of the cell, separating the contents from the rest of the cytoplasm, (ii) the fusion of the resultant vesicle with a lysosome and the subsequent degradation of the vesicle contents. The term autophagy may also refer to one of the mechanisms by which a starving cell re-allocates nutrients from unnecessary processes to more essential processes. The deacetylation can be achieved by three classes of compounds that (i) deplete the cytosolic pool of acetyl coenzyme A (AcCoA; the sole donor of acetyl groups), (ii) inhibit acetyl transferases (a group of enzymes that acetylate lysine residues in an array of proteins) or (iii) that stimulate the activity of deacetylases and hence reverse the action of acetyl transferases.(1) As used herein, the term “inhibitor” refers to any compound or treatment that reduces or blocks the activity of the target protein (e.g. an enzyme). The term also includes inhibitors of the expression of the target protein. As used herein, the phrase “inhibiting the activity” of a gene product refers to a decrease in a particular activity associated with the gene product. Examples of inhibited activity include, but are not limited to, decreased translation of mRNA, decreased signal transduction by polypeptides or proteins and decreased catalysis by enzymes. Inhibition of activity can occur, for example, through a reduced amount of activity performed by individual gene products, through a decreased number of gene products performing the activity, or through any combination thereof. If a gene product enhances a biological process {e.g. autophagy), “inhibiting the activity” of such a gene product will generally inhibit the process. Conversely, if a gene product functions as an inhibitor of a biological process, “inhibiting the activity” of such a gene product will generally enhance the process.

In some embodiments, the caloric restriction mimetic is an inhibitor of mitochondrial pyruvate carrier complex (MPC). An example of a pharmacological inhibitor includes alpha-cyanocinnamate derivative UK5099 (2-Cyano-3-(1-phenyl-1H-indol-3-yl)-2-propenoic acid).

In some embodiments, the caloric restriction mimetic is an inhibitor of mitochondrial carnitine palmitoytransferase-1 (CTP1). An example of a pharmacological inhibitor includes perhexiline (PHX). In some embodiments, the inhibitor is an inhibitor of CTP1c expression.

In some embodiment, the caloric restriction mimetic is an inhibitor of mitochondrial citrate carrier (CiC). An example of a pharmacological inhibitor includes benzenetricarboxylate (BTC).

In some embodiment, the caloric restriction mimetic is an inhibitor of ATP-citrate lyase (ACLY). An example of a pharmacological inhibitor includes hydroxycitrate. Other examples include this described in WO1993022304A1, U.S. Pat. Nos. 5,447,954, 6,414,002 US20030087935, and US20030069275. Other known inhibitors include (R,S)—S-(3,4-dicarboxy-3-hydroxy-3-methyl-butyl)-CoA, S-carboxymethyl-CoA and SB-204990 ((3R,5S)-re1-5-[6-(2,4-Dichlorophenyl)hexyl]tetrahydro-3-hydroxy-2-oxo-3-furanacetic acid) and BMS-303141 (3,5-Dichloro-2-hydroxy-N-(4-methoxy[1,1′-biphenyl]-3-yl)-benzenesulfonamide).

In some embodiments, the caloric restriction mimetic is an EP300 acetyltransferase inhibitor. As used herein the term EP300 refers to the “E1A binding protein p300” protein which functions as histone acetyltransferase that regulates transcription via chromatin remodeling and is important in the processes of cell proliferation and differentiation. Examples of EP300 acetyltransferase inhibitors include but are not limited to aspirin, salicylate and C646 which has the following formula:

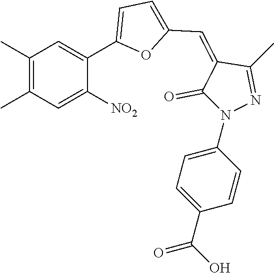

In some embodiments, the caloric restriction mimetic is an inhibitor of acyl-CoA synthetase short-chain family member 2 (ACCS2).

In some embodiments, the caloric restriction mimetic is spermidine or a metabolically stable analogue of spermidine. The term “spermidine”, as used herein, refers to the compound H2N—(CH2)3-NH(CH2)4-NH2. The term “metabolically stable analogue of spermidine”, as used herein, refers to compounds which are structurally related to spermidine, but which are substantially not metabolized in vivo, including, but not limited to, (1-methylspermidine) H2N—CH(CH3)-(CH2)2-NH(CH2)4-NH2. Such metabolically stable analogues may include spermidine analogues which are not substantially susceptible to enzymes that metabolize polyamines.

In one embodiment, the CRM is not a FAK (focal adhesion kinase) inhibitor.

As used herein, the term “combination” is intended to refer to all forms of administration that provide a first drug together with a further (second, third . . . ) drug. The drugs may be administered simultaneously, separately or sequentially and in any order. Drugs administered in combination have biological activity in the subject to which the drugs are delivered. Within the context of the invention, a combination thus comprises at least 3 different drugs, and wherein the first drug is a chemotherapeutic agent, the second drug is an immunotherapeutic agent (e.g. an immune checkpoint inhibitor) and the third drug is a caloric restriction mimetic, as previously described. In some instance, the combination of the present invention results in the synthetic lethality of the cancer cells. In some embodiments, the caloric restriction mimetic is administered to the patient before the administration of the chemotherapeutic agent and the immunotherapeutic agent.

In some embodiments, the patient is first administered with at least one cycle (C1) of chemotherapy with the caloric restriction mimetic followed by administration of at least one cycle (C2) of immunotherapy. As used herein, the term “cycle” refers to a period of time during the treatment is administered to the patient. Typically, in cancer therapy a cycle of therapy is followed by a rest period during which no treatment is given. Following the rest period, one or more further cycles of therapy may be administered, each followed by additional rest periods. In some embodiments, cycle (C1) comprises administering a dose of the caloric restriction mimetic daily or every 2, 3, 4, or 5 days. In some embodiments, the caloric restriction mimetic is administered continuously (i.e. every day) during cycle (C1). In some embodiments, cycle (C1) comprises administering a dose of the chemotherapeutic agent daily or every 2, 3, 4, or 5 days. In some embodiments, cycle (C1) can start with administration of the caloric restriction mimetic followed by administration of the chemotherapeutic agent. In some embodiments, the administration of a dose of the caloric restriction mimetic is alternated with the administration of a dose of the chemotherapeutic agent. Typically cycle (C1) can last one or more days, but is usually one, two, three or four weeks long. In some embodiments cycle (C1) is repeated at least 2, 3, 4, 5, 6, 7, 8, 9 or 10 times before administering cycle (C2). In some embodiments, cycle (C2) consists in administering a dose of the immune checkpoint inhibitor weekly or every, 2, 4, or 5 weeks. In some embodiments, at the end of cycle (C1), the tumor infiltration of CD8+ T cells and or Treg cells is(are) quantified as described above. Then if the infiltration of CD8+ T cells increases and/or the infiltration of Tregs decreases after cycle (C1) then the patient is administered with cycle (C2). If the infiltration of CD8+ T cells and/or the infiltration of Tregs decreases after the cycle (C1) is not modified, the physician can decide to repeat cycle (C1).

In a particular embodiment, the invention relates to a composition comprising at least one a caloric restriction mimetic, as previously described, for use in a method for treating a cancer, as previously described. The method according to such embodiment further comprises administrating at least one chemotherapeutic agent and at least one immune-checkpoint inhibitor as previously described.

In one variant the composition comprising at least one CRM is simultaneously administrated in a combined preparation with the at least one chemotherapeutic and the at least one immune-checkpoint inhibitor.

In one variant the composition comprising at least one CRM is administrated sequentially, preferably prior to the administration of the at least one chemotherapeutic and the at least one immune-checkpoint inhibitor. In one variant the composition comprising at least one CRM is administrated from about 5 minutes to about 72 hours, from about 5 minutes to about 48 hours, from about 30 minutes to about 48 hours, from about 15 minutes to about 12 hours, from about 15 minutes to about 8 hours prior to the administration of the at least one chemotherapeutic and/or the at least one immune-checkpoint inhibitor.

In one embodiment, the method comprises:
a) a first administration of the composition comprising at least one CRM as previously described followed by subsequent daily administrations of the same; then
b) a first administration the at least one chemotherapeutic agent at least 12 hours, typically 24 our 48 hours past the first administration according to step (a), followed by subsequent daily or weekly administrations of the chemotherapy as defined by the medical protocols.
c) a first administration of at least one immune checkpoint inhibitor 12 hours, typically 24 our 48 hours past the first administration according to step (a), followed by subsequent daily or weekly administrations of the at least one immune checkpoint inhibitor as defined by the medical protocols.
   One skilled in the art can define whether the first and/or the subsequent administrations according to (b) or (c) are to be administrated simultaneously, sequentially or intermittently.

In a particular embodiment, the invention relates to a composition comprising at least one a caloric restriction mimetic, as previously described, for use in a method for treating a cancer, as previously described. The method according to such embodiment further comprises administrating at least one radiotherapy and at least one immune-checkpoint inhibitor as previously described.

In one embodiment, the composition according to the invention comprises at least one CRM selected from list N for use in a method comprising the administration of at least one chemotherapeutic agent selected from A and at least one immune checkpoint inhibitor selected from list I.

In one embodiment, the composition according to the invention comprises at least one CRM selected from list O for use in a method comprising the administration of at least one chemotherapeutic agent selected from A and at least one immune checkpoint inhibitor selected from list I.

In one embodiment, the composition according to the invention comprises at least one CRM selected from list P for use in a method comprising the administration of at least one chemotherapeutic agent selected from C and at least one immune checkpoint inhibitor selected from list L.

In one embodiment, the composition according to the invention comprises at least one CRM selected from list O for use in a method comprising the administration of at least one chemotherapeutic agent selected from B and at least one immune checkpoint inhibitor selected from list J.

In one embodiment, the composition according to the invention comprises at least one CRM selected from list O for use in a method comprising the administration of at least one chemotherapeutic agent selected from B and at least one immune checkpoint inhibitor selected from list K.

In one embodiment, the composition according to the invention comprises at least one CRM selected from list O for use in a method comprising the administration of at least one chemotherapeutic agent selected from C and at least one immune checkpoint inhibitor selected from list K.

In one embodiment, the composition according to the invention comprises at least one CRM selected from list P for use in a method comprising the administration of at least one chemotherapeutic agent selected from D and at least one immune checkpoint inhibitor selected from list L.

In one embodiment, the composition according to the invention comprises at least one CRM selected from list P for use in a method comprising the administration of at least one chemotherapeutic agent selected from E and at least one immune checkpoint inhibitor selected from list L.

In one embodiment, the composition according to the invention comprises at least one CRM selected from list P for use in a method comprising the administration of at least one chemotherapeutic agent selected from F and at least one immune checkpoint inhibitor selected from list L.

In one embodiment, the composition according to the invention comprises at least one CRM selected from list P for use in a method comprising the administration of at least one chemotherapeutic agent selected from G and at least one immune checkpoint inhibitor selected from list L.

In one embodiment, the composition according to the invention comprises at least one CRM selected from list P for use in a method comprising the administration of at least one chemotherapeutic agent selected from H and at least one immune checkpoint inhibitor selected from list L.

In one embodiment, the composition according to the invention comprises at least one CRM selected from list Q for use in a method comprising the administration of at least one chemotherapeutic agent selected from D and at least one immune checkpoint inhibitor selected from list L.

In one embodiment, the composition according to the invention comprises at least one CRM selected from list Q for use in a method comprising the administration of at least one chemotherapeutic agent selected from E and at least one immune checkpoint inhibitor selected from list L.

In one embodiment, the composition according to the invention comprises at least one CRM selected from list Q for use in a method comprising the administration of at least one chemotherapeutic agent selected from F and at least one immune checkpoint inhibitor selected from list L.

In one embodiment, the composition according to the invention comprises at least one CRM selected from list Q for use in a method comprising the administration of at least one chemotherapeutic agent selected from G and at least one immune checkpoint inhibitor selected from list L.

In one embodiment, the composition according to the invention comprises at least one CRM selected from list Q for use in a method comprising the administration of at least one chemotherapeutic agent selected from H and at least one immune checkpoint inhibitor selected from list L.

In one embodiment, the composition according to the invention comprises at least one CRM selected from list R for use in a method comprising the administration of at least one chemotherapeutic agent selected from D and at least one immune checkpoint inhibitor selected from list L.

In one embodiment, the composition according to the invention comprises at least one CRM selected from list R for use in a method comprising the administration of at least one chemotherapeutic agent selected from E and at least one immune checkpoint inhibitor selected from list L.

In one embodiment, the composition according to the invention comprises at least one CRM selected from list R for use in a method comprising the administration of at least one chemotherapeutic agent selected from F and at least one immune checkpoint inhibitor selected from list L.

In one embodiment, the composition according to the invention comprises at least one CRM selected from list R for use in a method comprising the administration of at least one chemotherapeutic agent selected from G and at least one immune checkpoint inhibitor selected from list L.

In one embodiment, the composition according to the invention comprises at least one CRM selected from list R for use in a method comprising the administration of at least one chemotherapeutic agent selected from H and at least one immune checkpoint inhibitor selected from list L.

In one embodiment, the composition according to the invention comprises at least one CRM selected from list P for use in a method comprising the administration of at least one chemotherapeutic agent selected from D and at least one immune checkpoint inhibitor selected from list L.

In one embodiment, the method does not comprise the administration of a FAK (focal adhesion kinase) inhibitor. In some embodiments, the invention does not concern the following examples of FAK inhibitors: VS-4718, VS-5095, and related compounds, or a pharmaceutically acceptable salt thereof. In some embodiments, the invention does not concern compounds VS-4718, VS-5095, and related compounds described in PCT/US2010/045359 and US20110046121. In some embodiments, the invention does not concern a compound of Formula (I-a) which is also referred to as VS-4718. In some embodiments, the invention does not concern a compound of Formula (I-b) which is also referred to as VS-5095. In some embodiments, the invention does not concern the FAK inhibitor which is a compound of Formula (I-a) or (I-b):

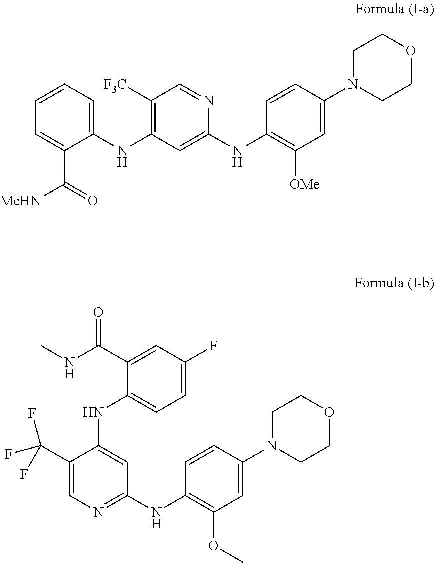 [SMILES: CNC(=O)c1ccccc1Nc1cc(Nc2ccc(N3CCOCC3)cc2OC)ncc1C(F)(F)F ; CNC(=O)c1cc(F)ccc1Nc1cc(Nc2ccc(N3CCOCC3)cc2OC)ncc1C(F)(F)F]

In some embodiments, the invention does not concern the following examples of FAK inhibitors: GSK2256098 and related compounds, or a pharmaceutically acceptable salt thereof. In some embodiments, the invention does not concern GSK2256098 and related compounds are described in US20100113475, US20100317663, US20110269774, US20110207743, US20140155410, and US2014010713. In some embodiments, the invention does not concern the FAK inhibitor which is a compound of Formula (I-c1), (I-c2), (I-c3), (I-c4), or (I-c5):

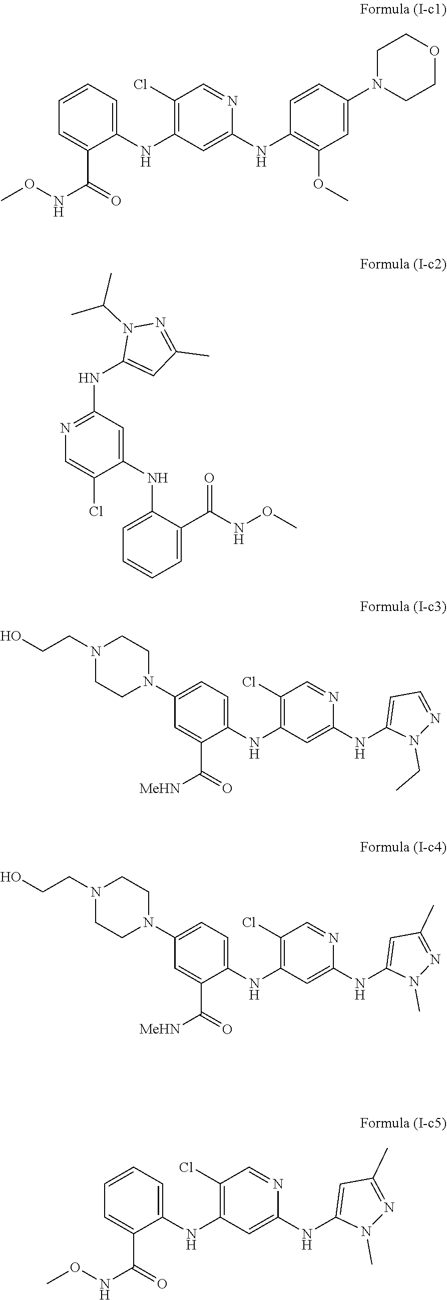 [SMILES: CONC(=O)c1ccccc1Nc1cc(Nc2ccc(N3CCOCC3)cc2OC)ncc1Cl ; CONC(=O)c1ccccc1Nc1cc(Nc2cc(C)nn2C(C)C)ncc1Cl ; CCn1nccc1Nc1cc(Nc2ccc(N3CCN(CCO)CC3)cc2C(=O)NC)c(Cl)cn1 ; CNC(=O)c1cc(N2CCN(CCO)CC2)ccc1Nc1cc(Nc2cc(C)nn2C)ncc1Cl ; CONC(=O)c1ccccc1Nc1cc(Nc2cc(C)nn2C)ncc1Cl]

In some embodiments, the invention does not concern the following examples of FAK inhibitors: VS-6063, VS-6062, and related compounds, or a pharmaceutically acceptable salt thereof (e.g. VS-6063 hydrochloride, VS-6062 hydrochloride). In some embodiments, the invention does not concern VS-6063, VS-6062, and related compounds which are also disclosed in, e.g. U.S. Pat. No. 7,928,109, EP1578732, PCT/IB2004/202744, PCT/IB2003/005883, PCT/IB 2005/001201, and PCT/IB2006/003349. In some embodiments, the invention does not concern VS-6063 which is also known as a compound of Formula (I-d), defactinib and PF-04554878. In some embodiments, the invention does not concern VS-6062 which is also known as a compound of Formula (I-d) and PF-00562271. In some embodiments, the invention does not concern the FAK inhibitor which is a compound of Formula (I-d) or (I-e):

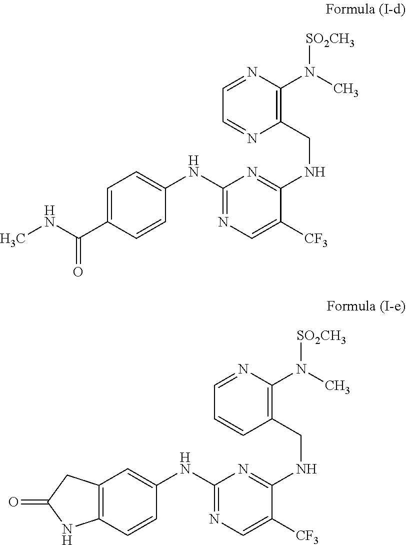

In some embodiments, the invention does not concern the following examples of FAK inhibitors of formula (I-f), formula (I-g), and related compounds, or a pharmaceutically acceptable salt thereof. In some embodiments, the invention does not concern a compound of Formula (I-f) and related compounds which are described in U.S. Pat. No. 8,569,298. In some embodiments, the invention does not concern the FAK inhibitor which is 2-[[2[(1,3-dimethylpyrazol-4-yl)amino]-5-(trifluoromethyl)-4-yridyl]amino]-5-fluoro-N-methoxy benzamide, or a compound of Formula (I-f):

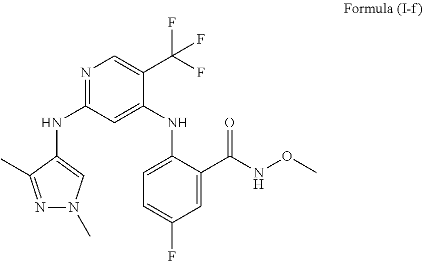

In some embodiments, the invention does not comprise the administration of the FAK inhibitor which is BI 853520.

As used herein, the term “therapeutically effective combination” as used herein refers to an amount or dose of each drugs (i.e. the chemotherapeutic agent, the immunotherapeutic agent and the caloric restriction mimetic) that is sufficient to treat the disease (e.g. cancer). A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens of drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an antibody of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the agent of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

In one embodiment, the composition for use according to the invention comprises at least one CRM as previously described in an amount ranging from 200 mg to 1.5 g, typically from 400 mg to 1.2 g, preferably from 600 to 1000 mg, even more preferably from 600 mg to 800 mg. In one typical embodiment, the CRM is hydroxycitrate in an amount ranging from 400 to 1000 mg, preferably from 600 to 900 mg. In one typical embodiment, the CRM is alpha-lipoic acid in an amount ranging from 400 to 700 mg, preferably from 500 to 700 mg.

In one embodiment, the composition for use according to the invention is administrated at least once a day, typically at least twice a day. In one embodiment, the composition for use according to the invention comprises hydroxycitrate and/or alpha-lipoic acid and is administrated at least once a day, typically at least twice a day, preferably at least three times a day.

Typically, the drug is administered to the subject in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a subject, the composition will be formulated for administration to the subject. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

A further object of the present to a kit comprising (a) a chemotherapeutic agent, (b) an immunotherapeutic agent and (c) a caloric restriction mimetic. Kits typically include a label indicating the intended use of the contents of the kit and instructions for use. The term label includes any writing, or recorded material supplied on or with the kit, or which otherwise accompanies the kit. In some embodiments, the invention is directed to a kit for treating a cancer.

A further object of the present invention relates to a method of treating cancer in a patient in need thereof comprising administering to the patient a therapeutically effective combination of chemotherapy and/or immunotherapy with a caloric restriction mimetic, wherein administration of the combination results in enhanced therapeutic efficacy relative to the administration of the chemotherapy and/or immunotherapy alone.

A further object of the present invention relates to a method of treating cancer in a patient in need thereof comprising administering to the patient a therapeutically effective combination consisting of an immune checkpoint inhibitor, a chemotherapeutic agent, and a caloric restriction mimetic wherein administration of the combination results in enhanced therapeutic efficacy relative to the administration of the immune checkpoint inhibitor alone.

As used herein, the expression “enhanced therapeutic efficacy,” relative to cancer refers to a slowing or diminution of the growth of cancer cells or a solid tumor, or a reduction in the total number of cancer cells or total tumor burden. An “improved therapeutic outcome” or “enhanced therapeutic efficacy” therefore means there is an improvement in the condition of the patient according to any clinically acceptable criteria, including, for example, decreased tumor size, an increase in time to tumor progression, increased progression-free survival, increased overall survival time, an increase in life expectancy, or an improvement in quality of life. In particular, “improved” or “enhanced” refers to an improvement or enhancement of 1%, 5%, 10%, 25% 50%, 75%, 100%, or greater than 100% of any clinically acceptable indicator of therapeutic outcome or efficacy.

A further object of the present invention relates to a method for enhancing the potency of an immune checkpoint inhibitor administered to a patient as part of a treatment regimen, the method comprising administering to the patient a pharmaceutically effective amount of the immune checkpoint inhibitor in combination with a caloric restriction mimetic and a chemotherapeutic agent.

As used herein, the expression “enhancing the potency of an immune checkpoint” refers to the ability of the combined administration of the caloric restriction mimetic with the chemotherapeutic agent to increase the ability of the immune checkpoint inhibitor to enhance the proliferation, migration, persistence and/or cytotoxic activity of CD8+ T cells. The ability of the immune checkpoint inhibitor to enhance T CD8 cell killing activity may be determined by any assay well known in the art.

The invention will be further illustrated by the following figures and examples. However, these examples and figures should not be interpreted in any way as limiting the scope of the present invention.

### FIGURES

FIG. 1. Fasting improves tumor growth control in response to chemo-immunotherapy. (A) Experimental design Immunocompetent mice were engrafted subcutaneously with syngeneic fibrosarcoma (MCA205) cells. One week later, once tumor was palpable, mice underwent a 48h fasting (no food, NF) before receiving mitoxantrone (MTX)-based chemotherapy. A combination of two immune checkpoint inhibitors (ICIs), anti-PD-1 plus anti-CTLA-4, was later administered 8, 12 and 16 days post-chemotherapy. Tumor growth and survival were monitored every 2-3 days until day 50. (B) Individual tumor growth curves of mice treated with PBS, MTX and MTX+NF. (C) Individual tumor growth curves of mice treated with MTX+ICIs or MTX+ICIs+NF. (D) Mean tumor growth (n=9 per treatment group). (E) Comparison of the tumor volumes at day 24 post-MTX in alive mice of the different treatment groups. (F) Comparison of the tumor volumes at day 29 post-MTX in alive mice treated with MTX+ICIs versus MTX+ICIs+NF. Differences between tumor sizes were considered significant when p value<0.05. *p<0.05, **p<0.01, ***p<0.001, ****p<0.0001.

FIG. 2. Aspirin improves the efficacy of chemo-immunotherapy. (A) Experimental design Immunocompetent mice were engrafted subcutaneously with syngeneic fibrosarcoma (MCA205) cells. One week later, once tumors were palpable, mice received one intraperitoneal injection of aspirin (Asp) at day −1 and 0 post-mitoxantrone (MTX). Starting from day 2, aspirin was injected once a day for 5 days per week. A combination of two immune checkpoint inhibitors (ICIs), anti-PD-1 plus anti-CTLA-4, was later administered 8, 12 and 16 days post-chemotherapy. Tumor growth was monitored every 2-3 days until day 50. (B) Individual tumor growth curves of mice treated with PBS, MTX and MTX+Asp. (C) Individual tumor growth curves of mice treated with MTX+ICIs or MTX+ICIs+Asp. (D) Mean tumor growth (n=8 per treatment group). (E) Comparison of the tumor volumes at day 22 post-MTX in alive mice of the different treatment groups. (F) Comparison of the tumor volumes at day 35 post-MTX in alive mice treated with MTX+ICIs versus MTX+ICIs+Asp. Differences between tumor sizes were considered significant when p value<0.05. *p<0.05, **p<0.01, ****p<0.0001.

FIG. 3. Hydroxycitrate enhances tumor growth control mediated by chemo-immunotherapy. (A) Experimental design Immunocompetent mice were engrafted subcutaneously with syngeneic fibrosarcoma (MCA205) cells. One week later, once tumor was palpable, hydroxycitrate (HC) was added to mouse drinking water daily, starting from day −1 until day 45 post-mitoxantrone (MTX)-based treatment. A combination of two immune checkpoint inhibitors (ICIs), anti-PD-1 plus anti-CTLA-4, was later administered 8, 12 and 16 days post-chemotherapy. Tumor growth was monitored every 2-3 days until day 50. (B) Individual tumor growth curves of mice treated with PBS, MTX and MTX+HC. (C) Individual tumor growth curves of mice treated with MTX+ICIs or MTX+ICIs+HC. (D) Mean tumor growth (n=9 per treatment group). (E) Comparison of the tumor volumes at day 22 post-MTX in alive mice of the different treatment groups. (F) Comparison of the tumor volumes at day 31 post-MTX in alive mice treated with MTX+ICIs versus MTX+ICIs+HC. Differences between tumor sizes were considered significant when p value<0.05. *p<0.05, ***p<0.001, ****p<0.0001.

FIG. 4. Spermidine significantly improves tumor outcome upon chemo-immunotherapy. (A) Experimental design Immunocompetent mice were engrafted subcutaneously with syngeneic fibrosarcoma (MCA205) cells. One week later, once tumor was palpable, mice received one intraperitoneal injection of spermidine (Spd) at day −1 and 0 post-mitoxantrone (MTX). Starting from day 2, spermidine was injected once a day every 2 to 3 days until day 45. A combination of two immune checkpoint inhibitors (ICIs), anti-PD-1 plus anti-CTLA-4, was later administered 8, 12 and 16 days post-chemotherapy. Tumor growth was monitored every 2-3 days until day 50. (B) Individual tumor growth curves of mice treated with PBS, MTX and MTX+Spd. (C) Individual tumor growth curves of mice treated with MTX+ICIs or MTX+ICIs+Spd. (D) Mean tumor growth (n=9 per treatment group). (E) Comparison of the tumor volumes at day 22 post-MTX in alive mice of the different treatment groups. (F) Comparison of the tumor volumes at day 31 post-MTX in alive mice treated with MTX+ICIs versus MTX+ICIs+Spd. (G) Mice cured from MC205 fibrosarcoma following MTX+ICIs+Spd treatment were rechallenged subcutaneously with MCA205 on one flank and with antigenically unrelated TC1 lung carcinoma cells into the contralateral flank (n=4).

Appearance of each tumor was monitored and displayed as a Kaplan-Meier curve. Differences between tumor sizes were considered significant when p value<0.05. *p<0.05, **p<0.01, ***p<0.001, ****p<0.0001.

FIG. 5. The benefit of ICIs in combination with chemotherapy and caloric restriction mimetics results from PD-1 rather than CTLA-4 blockade. (A) Mean tumor growth (n=8 per treatment group). (B) Individual tumor growth curves of mice treated with MTX+Spd plus either the combination of both anti-PD-1 and anti-CTLA-4 or each ICI alone. (C) Comparison of the tumor volumes at day 24 post-MTX in alive mice treated with MTX+Spd+both ICIs versus MTX+Spd+anti-PD-1 alone or MTX+Spd+anti-CTLA-4 alone. A clear trend in favor of the benefit of PD-1 blockade to the MTX+Spd combinatory treatment, rather than blockade of CTLA-4, was observed. ICI, immune checkpoint inhibitor; MTX, mitoxantrone; Spd, spermidine.

FIG. 6. CRMs improve MTX+ICBs based-therapy. MTX and ICBs (anti-PD-1+anti-CTLA-4) combination efficacy can be further enhanced by HC, Spd or NF. WT 7 weeks old C57B1/6 mice were subcutaneously injected with MCA205 WT fibrosarcoma cells. When tumors became palpable, mice underwent two days of fasting (d-2 to d0). Continuous treatments with HC in drinking water or Spd i.p injections began the day after (d-1), followed by chemotherapy with MTX (d0). ICBs i.p injections were administered at days 8, 12 and 16 post-chemotherapy. Individual tumor growth curves; (A) tumor volumes (mm³) at day 24 post-chemotherapy (or last tumor measurement when mice were sacrificed); and (B and C) survival curves. Ordinary one-way ANOVA was realized for tumor volumes at day 24 post-chemotherapy (A) and Log-rank (Mantel-Cox) test was realized for survival curves (B and C).

FIG. 7. CRMs improve OXA+anti-PD-1 based-therapy. OXA and anti-PD-1 combination efficacy can be further enhanced by HC, Spd or NF. WT 7-11 weeks old C57B1/6 mice were subcutaneously injected with MCA205 WT fibrosarcoma cells. When tumors became palpable, mice underwent two days of fasting (d-2 to d0). Continuous treatments with HC in drinking water or Spd i.p injections began the day after (d-1), followed by chemotherapy with OXA (d0). ICBs i.p injections were given at days 9, 13 and 17 post-chemotherapy. Individual tumor growth curves; (A) tumor volumes (mm³) at day 24 post-chemotherapy (or last tumor measurement when mice were sacrificed); and (B and C) survival curves.

The data shown represent a pool of two independent experiments sharing the groups PBS, OXA, OXA+HC, OXA+aPD-1, OXA+HC+aPD-1. Ordinary one-way ANOVA was realized for tumor volumes at day 24 post-chemotherapy (A) and Log-rank (Mantel-Cox) test was realized for survival curves (B and C).

FIG. 8. MTX impacts PD-L1 expression on CD45^{+/-} infiltrating cells.

(A-D) MTX alone or in combination with NF or HC stimulates the expression of PD-L1 on immune (CD45⁺) and tumor cells (CD45⁻). WT 9 weeks old C57B1/6 mice were subcutaneously injected with MCA205 WT fibrosarcoma cells. When tumors became palpable, mice underwent two days of fasting (d-2 to d0). HC treatment in drinking water began the day after (d-1), followed by chemotherapy with MTX (d0). 11 days post-chemotherapy, mice were sacrified and tumors were collected, dissociated, filtrated and stained with panel 3 antibodies. The results are represented as percentage among viable cells. In the tumor immune infiltrate: (A and B) MTX alone or in combination with NF or HC increases the percentage (A) of CD45⁻ PD-L1⁺ cells and the mean fluorescent intensity (B) of PD-L1 on CD45⁻ cells. (C and D) MTX alone or in combination with NF or HC increases the percentage (C) of CD45⁺ PD-L1⁺ cells and the mean fluorescent intensity (D) of PD-L1 on CD45⁻ cells. The data shown represent a pool of two independent experiments sharing all the groups. Statistical analysis were realized using ordinary one-way ANOVA. ****p<0.001, ***p<0.005, **p<0.01, *p<0.05.

### EXAMPLE

Methods:

Mouse strain and housing. Six- to 8-week-old wild-type female C57B1/6 mice were obtained from Envigo RMS SARL (Gannat, France). Animals were maintained in specific pathogen-free conditions in a temperature-controlled environment with 12 h light, 12 h dark cycles and received food and water ad libitum (unless precised otherwise). Animal experiments were in compliance with the EU Directive 63/2010 and approved by the Ethical Committee of the Cordeliers Research Center (Paris, France). All mouse experiments were randomized and blinded, and sample sizes were calculated to detect a statistically significant effect.

In vivo experimentations. Tumor engraftment was performed through subcutaneous injection of 3×10⁵ MCA205 fibrosarcoma tumor cells (in 100 μl PBS) in the right flank of the mice. Tumor volume was monitored using a digital caliper and calculated according to the formula: volume=length×width×height/8×4/3 pi. When tumor reached 20 mm³ on average, mice underwent fasting (48 hours without food but ad libitum access to water) or were given caloric restriction mimetics (CRMs) such as aspirin (Asp; 10 mg/kg i.p. in 200 μl phosphate buffered saline [PBS] five times per week), hydroxycitrate (HC; 5 mg/ml in drinking water daily) or spermidine (Spd; 50 mg/kg i.p. in 200 μl Earle's balanced salt solution three times per week), or were treated with mitoxantrone (MTX; 5.17 mg/kg i.p. in 200 μl PBS), or with the immune checkpoint inhibitors (ICIs) anti-PD-1 (10 mg/kg i.p. in 200 μl PBS) and/or anti-CTLA-4 (5 mg/kg i.p. in 200 μl PBS). Tumor size was carefully monitored up to 50 days post-MTX. Antitumor immunity induced by the treatment in cured mice was challenged by subcutaneously re-engrafting the same tumor (3×10⁵ syngeneic MCA205 fibrosarcoma cells) in one flank while an antigenically unrelated cancer (3×10⁵ syngeneic TC1 lung carcinoma cells) was implanted into the contralateral flank.

Hormone-Induced Orthotopic Mammary Tumor Model

Breast cancers were induced in young (7-weeks-old) female BALB/c mice by implantation of medroxyprogesterone acetate (MPA)-releasing pellets followed by gavage with the DNA damaging agent 7,12-Dimethylbenz[a]anthracene (DMBA) for the following 6 weeks. Note that the interval between the last DMBA injection and the manifestation of palpable breast cancer lesions is rather variable. When palpable tumors appeared, mice were randomized into the different experimental groups and treated with intraperitoneal injections of hydroxycitrate (100 mg/kg) at d-1 and d0 and/or intraperitoneal injections of 5.17 mg/kg of Mitoxantrone. Neutralizing anti-CD11b antibodies (clone M1/70, ref BE0007 from BioXCell™) or their isotype control (clone LTF-2, ref BE0090 from BioXCell™) were then injected at d-1, d0 and d7. Tumor growth was followed by calculating tumor surface (mm²) with the formula length×width.

Tissue Processing and Immunophenotyping of the Immune Infiltrate

3 or 11 days post-chemotherapy (d3 or d11), mice were euthanized and the tumors were withdrawn and placed in gentleMACS C tubes (ref 130-096-334 from Miltenyi Biotec™) previously filled with 1 ml of DMEM or RPMI medium, and immediately put on ice. After a mechanical (with scissors) and chemical digestion (thanks to the tumor dissociation kit and the gentleMACS Octo Dissociator, ref 130-096-730 and 130-096-427, respectively, from Miltenyi Biotec™), tumors were filtered (using MACS smartstrainers 70 uM, ref 130-110-916 from Miltenyi Biotec™), washed twice with PBS and distributed in a 96-wells round bottom plate.

Then, cells were stained with a live dead dye (ref L34959 from ThermoFisher Scientific™) and an FCblock receptor targeting antibody. For the surface stainings, several anti-mouse fluorochrome-coupled antibodies were employed, that were for 1) myeloid cells “staining 1”: anti-CD45 APC-Fire750 (clone 30E-11, ref 130154 Biolegend™), anti-Ly-6G PE (clone 1A8, ref 551461 BD™), anti-Ly-6C FITC (clone AL-21, ref 553104, BD™), anti-CD11b V450 (clone M1/70, ref 560455 BD™), anti-CD11c PE-Cy7 (clone HL3, ref 558470 BD™), anti-CD80 PerCP-Cy5.5 (16-10A1, ref 104722 Biolegend™), and anti-MHC-II APC (clone M5/114.15.2, ref 107614 Biolegend™) 2) T-cells “staining 2”: anti-CD3 APC (clone 17A2, ref 17-0032-82 eBioscience™), anti-CD8 PE (clone 53-6.7, ref 553032 BD™), anti-CD4 PerCP-Cy5.5 (clone RM4-5, ref 45-0042-82 eBioscience™), anti-CD25 PE-Cy7 (clone PC61.5, ref 25-0251-82 Invitrogen™), anti-ICOS BV421 (clone 7E.17G9, ref 564070 BD™) and anti-PD-1 APC-Fire750 (clone 29F.1Al2, ref 135240 Biolegend™) 3) PD-L1 expressing-cells “staining 4”: anti-CD45 AlexaFluor647 (clone 30E-11, ref 103-124 Biolegend™), anti-PD-L1 BV421 (clone MIHS, ref 564716 BD™) and anti-PD-L2 PE-Dazzle594 (clone TY25, ref 107215 Biolegend™) 4) NKT cells “staining 5”: anti-CD3 FITC (clone 17A2, ref 11-00-32-82 eBioscience™) and anti-NK1.1 PerCP-Cy5.5 (clone PK136, ref 551114 BD™). After the fixation and permeabilization of the cells (thanks to the Cytofix/Cytoperm kit, ref 554714 BD™ for stainings 1, 3, 4 and 5; and with the Foxp3/Transcription Factor kit, ref 00-5523-00 eBioscience™ for staining 2), intracellular stainings were performed using for “staining 2”: anti-FoxP3 FITC (clone FJK-16s, ref 11-5773-82 eBioscience™) and for “staining 3”: anti-IFNg APC (clone XMG1.2, ref 505-810 Biolegend™), anti-TNFα APC-Cy7 (clone MP6-XT22, ref 506344 Biolegend™) and anti-IL-2 PE-Dazzle 594 (clone JES6-5H4, ref 503-840 Biolegend™). Finally, cells were resuspended in FACS buffer and analyzed on a flow cytometer BD LSR II.

Statistical analyses. For tumor size comparison, Student unpaired t test or one-way ANOVA (Holm-Sidak) were performed. All statistical analyses were performed using GraphPad Prism version 6 for Windows (GraphPad Software, La Jolla, Calif., USA). Differences were considered significant when p value<0.05.

Results:

### Example 1

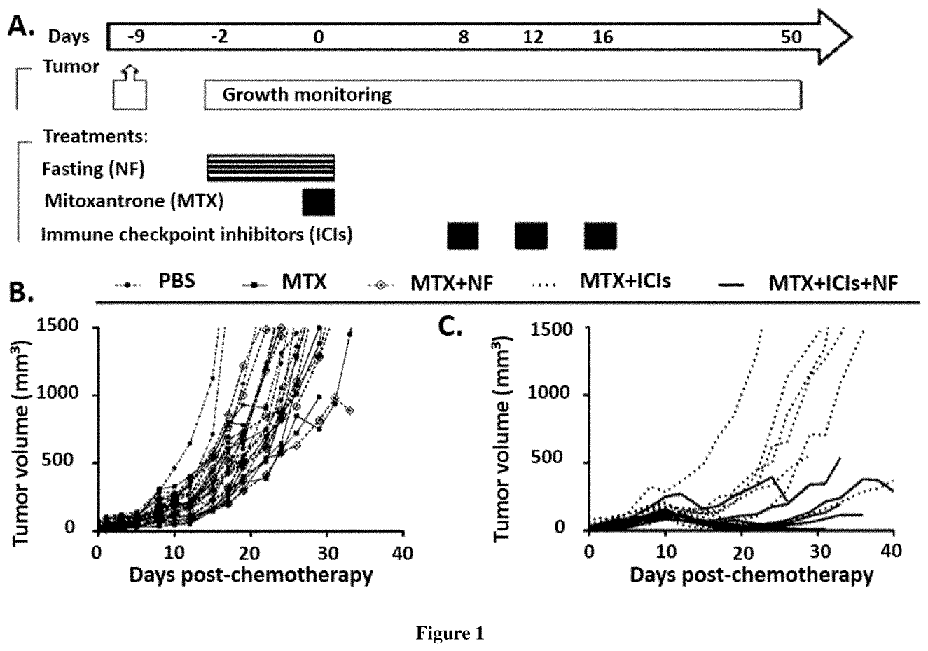

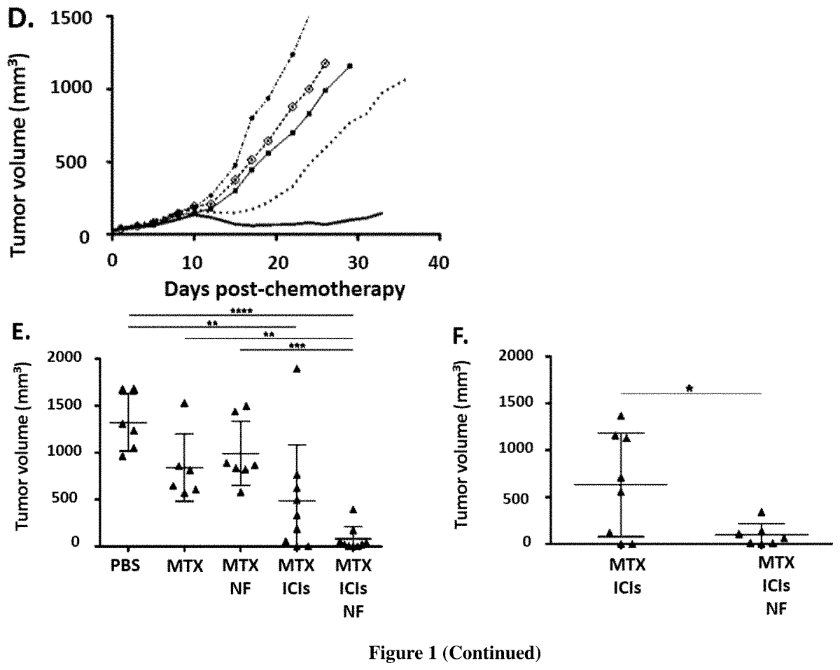

Immune checkpoint sensitization by the combination of chemotherapy and starvation. Immunocompetent mice bearing palpable syngeneic tumors (20 mm³ on average) developing in a subcutaneous location were first treated with systemic chemotherapy alone (mitoxantrone, MTX, injected intraperitoneally (i.p.), or PBS as a vehicle control) or in combination with a fasting regimen (48 hours, prior to chemotherapy) and then randomized in groups that either received immunotherapy (antibodies blocking CTLA-4 or PD-1) or isotype control antibodies, as indicated schematically in FIG. 1A. Tumor growth was monitored continuously. The combination therapy that yielded the most frequently tumor-free mice at the endpoint of the experiment (50 days after day 0 defined as the day of chemotherapy) consisted in the combined utilization of starvation, chemotherapy and immunotherapy. Complete responses leading to tumor eradication were either not seen at all or rare in any of the other groups (PBS controls, MTX plus isotypes, MTX plus HC, MTX plus immunotherapy) (FIG. 1B-F). Hence, a triple combination regimen (starvation, chemotherapy and immunotherapy) has a unique capacity to lead to the disappearance of cancers.

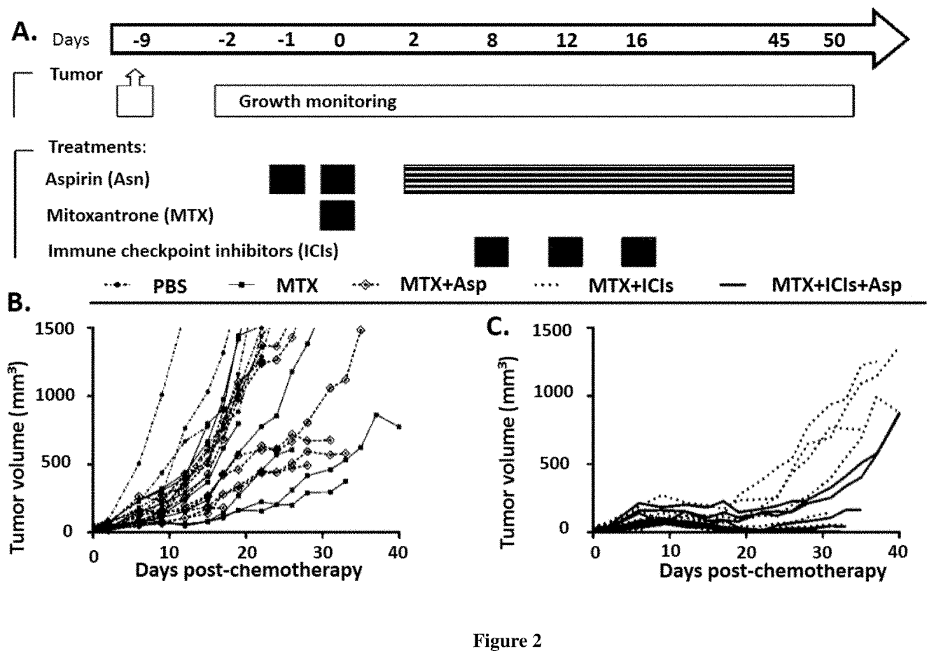

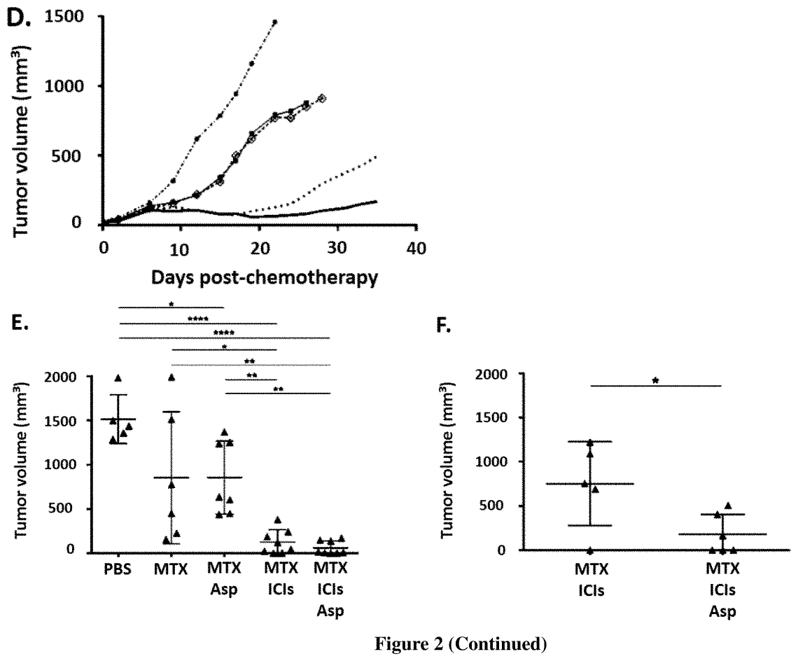

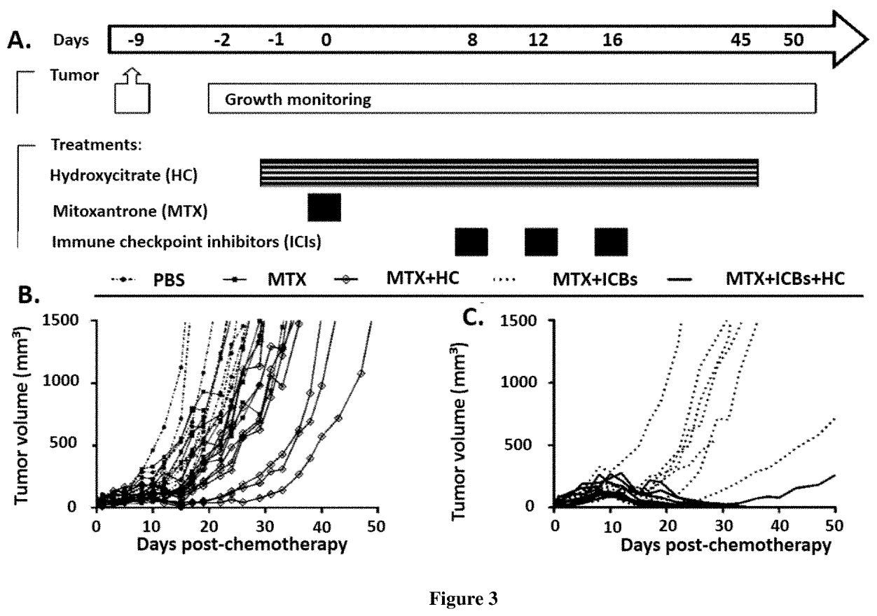

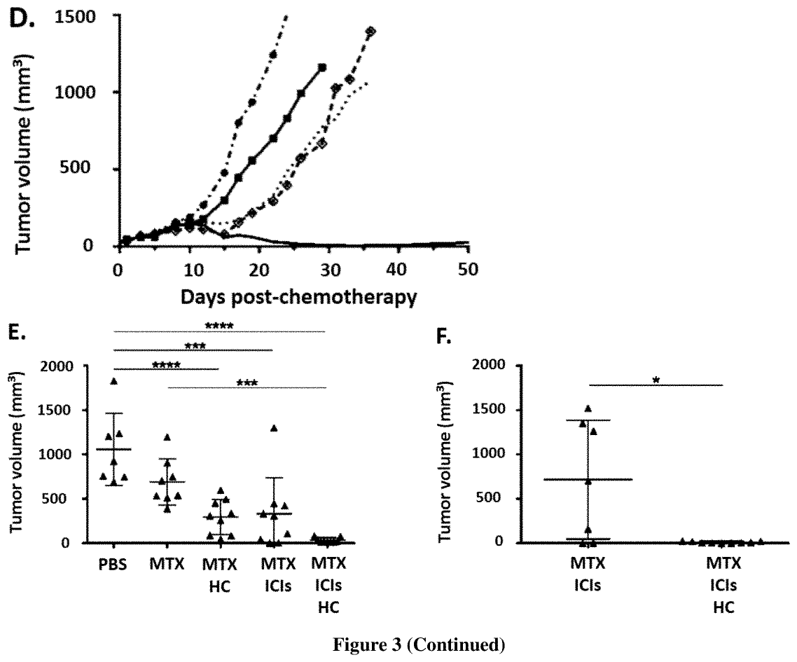

Immune checkpoint sensitization by the combination of chemotherapy and aspirin. Aspirin is a CRM in the sense that it induces autophagy in vivo through molecular pathways that resemble those induced by starvation.(3) We therefore performed an experiment in which starvation was replaced by five weekly i.p. injections of acetylsalicylate (the chemical name for aspirin), as indicated in FIG. 2A. The combination regimen that demonstrated superior efficacy in causing complete disappearance of subcutaneous cancers involved the utilization of aspirin, chemotherapy and immunotherapy (FIG. 2B-F). This combination regimen caused tumors to disappear below the detection threshold in 3 out of 6 cases at late timepoint (FIG. 2F). All other groups failed to yield regular tumor eradication as above (FIG. 2B-F). Altogether, we conclude that a triple combination regimen (aspirin, chemotherapy and immunotherapy) is particularly efficient at causing tumor regression. Immune checkpoint sensitization by the combination of chemotherapy and hydroxycitrate. As mentioned in the introduction, hydroxycitrate (HC) is a CRM.(2, 9) Consequently, we tested its use in the context of chemotherapy and immunotherapy. Since HC is orally available and non-toxic, we administered this agent in the drinking water, following the schedule indicated in FIG. 3A. Again, we found that the combination of HC, chemotherapy and immunotherapy was more efficient in reducing tumor growth than all other groups. Indeed, this triple combination caused complete responses at day 30 in all animals of the group with stable response in all but one (7 out of 8) mice (FIG. 3B-F). In conclusion, it appears that HC is particularly efficient at sensitizing mice to chemoimmunotherapy.

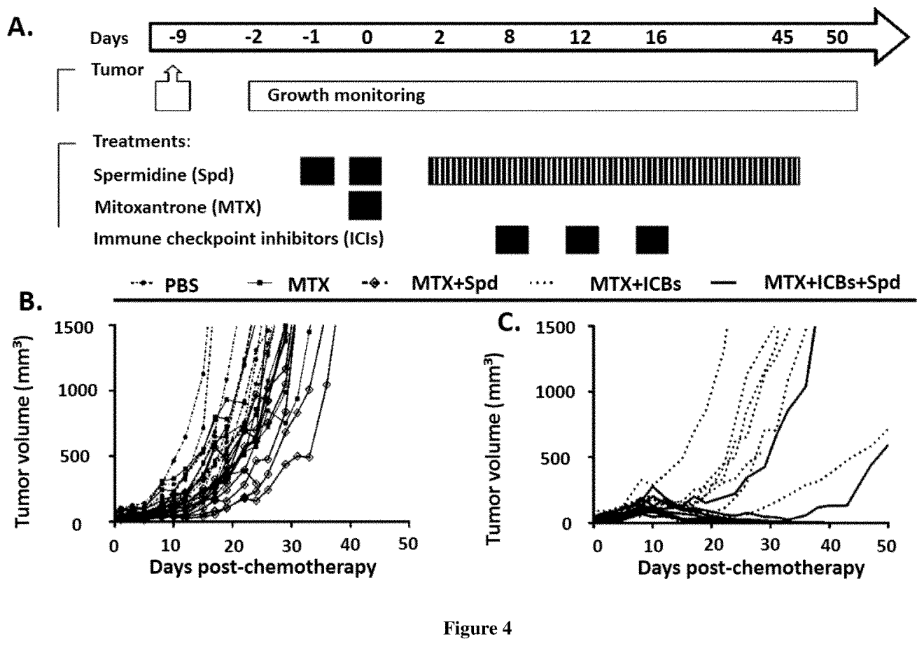

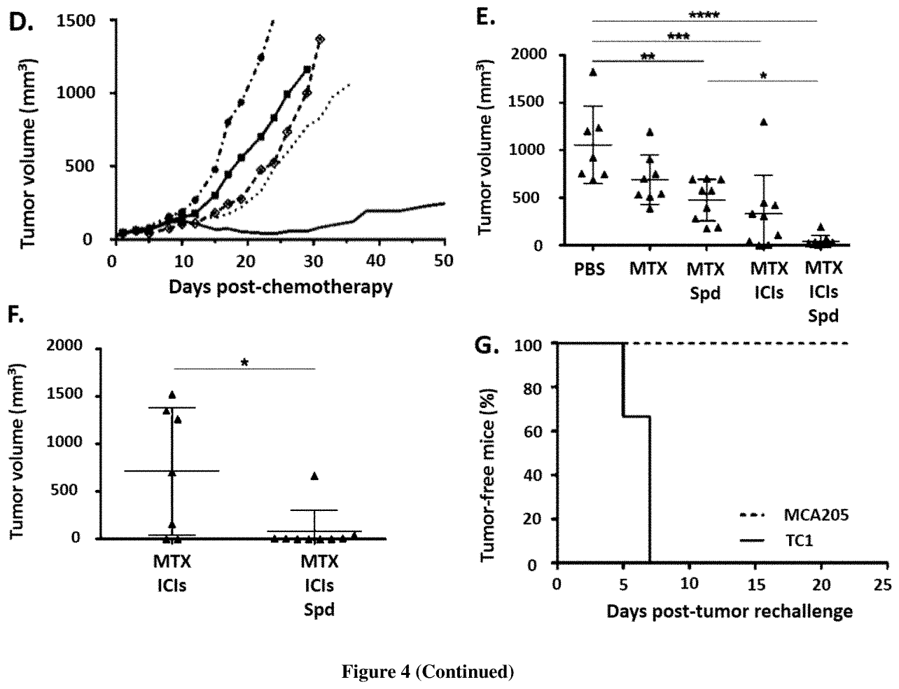

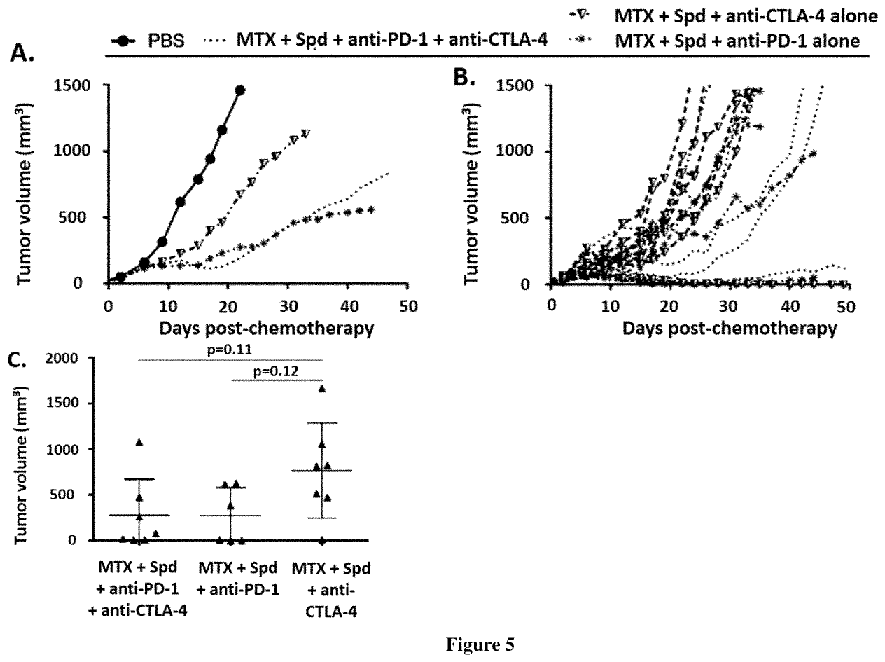

Immune checkpoint sensitization by the combination of chemotherapy and spermidine. Spermidine is yet another CRM with a favorable toxicology profile.(4, 27, 28) We administered spermidine via i.p. injection (3 times per week) together with chemotherapy or chemoimmunotherapy, as indicated in FIG. 4A. Spermidine was highly efficient at sensitizing to chemoimmunotherapy (chemotherapy plus dual CTLA-4/PD-1-targeting immunotherapy), leading to the cure of established tumor in 7 out of 9 mice (FIG. 4B-F). Importantly, rechallenge of cured mice with the same tumor that had been cured yielded the proof that a permanent cancer-protective immune response had been induced. Thus, reinjection of MCA205 cancer cells into mice that had been cured from MCA205 tumors did not led to the outgrowth of the neoplastic cells, while antigenically unrelated TC1 cancer cells injected into the opposite flank yielded tumors (FIG. 4G). In another, independent experiment, we compared dual immune checkpoint blockade (targeting both CTLA-4 and PD-1) with single immune checkpoint blockade (targeting either CTLA-4 or PD-1). Mice were first treated with the combination of MTX plus spermidine and then received three different kinds of immunotherapy (anti-CTLA-4 plus anti-PD-1, anti-PD-1 alone, anti-CTLA-4 alone). Complete cure was obtained in 3 out of 7 mice receiving anti-CTLA-4 plus anti-PD-1, in 3 out of 6 mice receiving anti-PD-1 alone and in 1 out of 7 animals receiving anti-CTLA-4 alone. These findings suggest that PD-1 blockade is more important for obtaining complete cure than CTLA-4 blockade (FIG. 5A-C). In conclusion, spermidine can sensitize cancers to a combination of chemotherapy and immunotherapy, the latter being based on dual immune checkpoint blockade (targeting CTLA-4 and the PD-1/PD-L1 interaction) or single immune checkpoint blockade (targeting the PD-1/PD-L1 interaction).

### Example 2

CD11b blockade interferes with the anticancer effects of hydroxycitrate combined with chemotherapy. The combination of the progesterone analogue medroxyprogesterone and repeated DNA damage by gavage with 2,4-dimethoxybenzaldehyde (DMBA) is highly efficient in inducing mammary carcinomas when administered to young female BALB/c mice (Data not shown). In this model, the combination of mitoxantrone (MTX)-based chemotherapy and the CRM hydroxycitrate (HC) is highly efficient in reducing tumor growth and prolonging mouse survival (Data not shown), much more so than MTX and HC alone.²⁶ These results were obtained in a ‘realistic’ setting in which treatments were started when the cancers could be diagnosed by palpation and hence reached a surface of 25 mm². Of note, repeated injections of a monoclonal antibody (M1/70) that blocks CD11b-dependent extravasation of myeloid cells¹⁵ significantly interfered with the tumor growth reduction by HC+MTX (Data not shown). Very similar results were obtained in a model of transplantable MCA205 fibrosarcoma developing on immunocompetent C57B1/6 mice (Data not shown). Again, the combination treatment with HC+MTX was more successful in reducing tumor growth and in prolonging survival than MTX alone, and the efficacy of this treatment was reduced by CD11b blockade (Data not shown).

Altogether, these results support the idea that myeloid cells (and presumably antigen-presenting cells) play a major role in the therapeutic efficacy of the combination of HC+MTX.

Effects of CRMs on the myeloid and lymphoid cancer immune infiltrate. Based on the aforementioned results, we decided to investigate the impact of fasting and two different CRMs (HC and spermidine) on the composition of the immune infiltrate of cancers in the context of MTX-based chemotherapy. At day 3 post-chemotherapy (that was optionally preceded by a 2-day fasting regimen or by a 24-hour treatment with HC or chronic supplementation with spermidine for up to 45-days, no major increments in the myeloid infiltrate were detected in response to fasting, HC or spermidine, perhaps because the immunosuppression mediated by MTX was still ongoing (Data not shown). Similarly, RNA-seq analyses of whole tumors failed to yield convincing evidence in favor of local immunostimulation by fasting, HC or spermidine at this time point (Data not shown). We therefore concentrated our effort on the characterization of the immune infiltrate at day 11 post-chemotherapy by immunophenotyping of CD45⁺ cells purified from the tumor bed. At this time point, MTX-treated cancers contained a higher density of CD45⁺ leukocytes, more so when the animals were starved or received HC (Data not shown). Of note, each of the co-treatments had a differential impact on the composition of the myeloid infiltrate. Thus, HC caused an increase in the granulocyte infiltration (phenotype: Ly6C⁺Ly6G^{hi}) (Data not shown) and a particular monocytic dendritic cell (mDC) subpopulation with activation markers (phenotype: Ly6G⁻Ly6C^{hi}CD11b⁺CD11c⁺CD80⁺MHC-II^{hi}) (Data not shown). Starvation led to the expansion of a less activated mDC subpopulation (phenotype: Ly6G⁻Ly6C^{hi}CD11b⁺CD11c⁺CD80⁺MHC-II^{lo}) (Data not shown). Spermidine caused the expansion of a macrophage subpopulation with an M1 phenotype (Ly6G⁻F4/80⁺CD11c⁻CD11b⁺CD38⁺) (Data not shown). The effects of starvation and CRMs were also determined at the level of the T lymphocyte infiltrate. When combined with MTX, NF (but neither HC nor spermidine) caused an increase in the density of total CD3⁺ and CD8⁺ T cell infiltrate (Data not shown). However, starvation or the CRMs failed to affect the T cell activation marker ICOS (Data not shown), the exhaustion marker PD-1 (Data not shown), the ratio of CD8⁺ over CD4⁺CD25⁺FoxP3⁺ regulatory T (Treg) cells (Data not shown) or the production of interferon-γ (IFNγ), tumor necrosis factor-α (TNFα) or interleukin-2 (IL-2) by T cells after stimulation with PMA/ionomycin (Data not shown).

Altogether, it appears that the changes induced by starvation or CRMs in the T cell compartment are relatively minor as compared to those affecting myeloid cells.

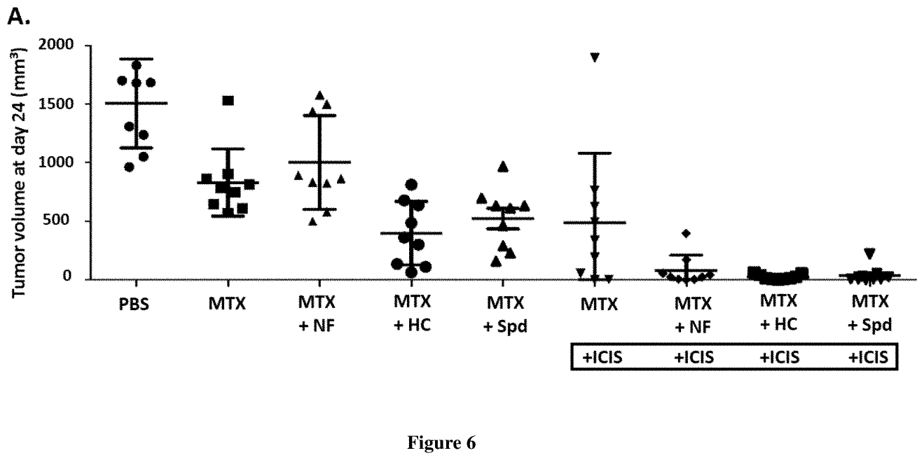

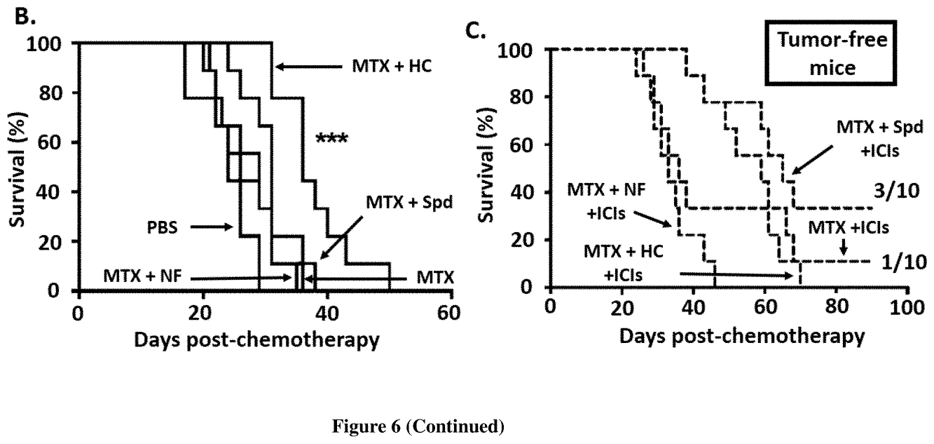

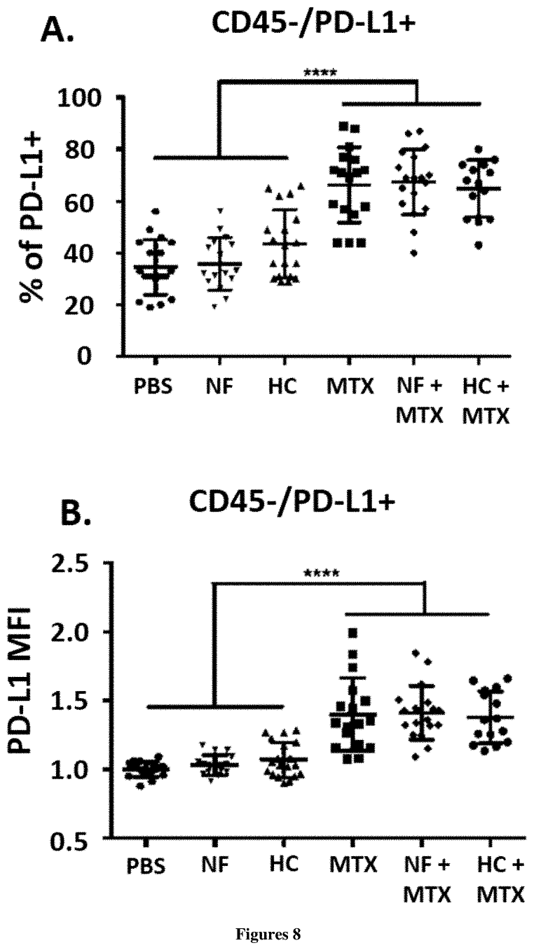

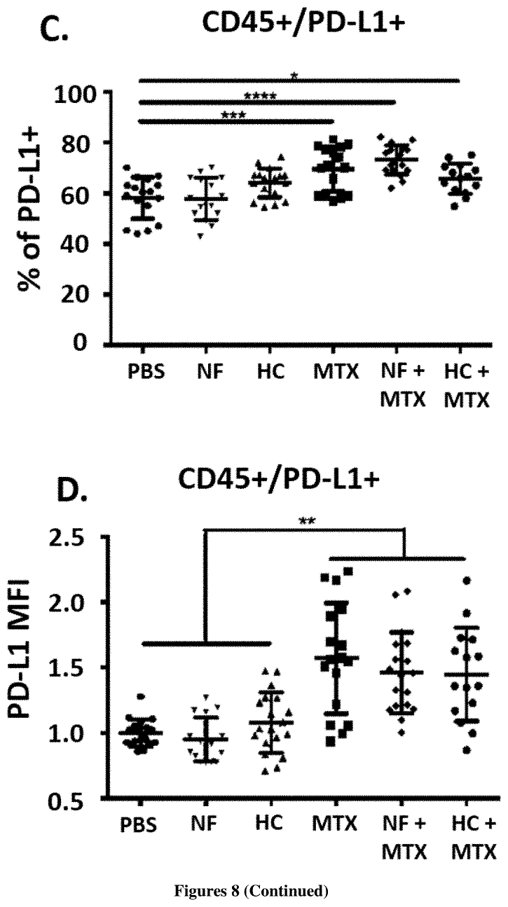

CRM-mediated sensitization to immune checkpoint blockade. We observed that treatment of MCA205 tumor-bearing mice with MTX induced the upregulation of PD-L1 both on non-leukocytes from the cancer (CD45⁻ cells, mostly malignant cells) (FIG. 8A, B) and in leukocytes expressing CD11b (FIG. 8C, D). This effect was not altered by co-treatment with starvation of HC (FIG. 8A-D). No changes were observed in the expression of PD-1 (Data not shown) and CTLA-4 (Data not shown) in response to MTX alone or together with fasting or CRMs. MTX also induced an increase in PD-L2 expression in CD45⁻ cells that was not affected by starvation nor by HC (Data not shown). Based on these results, we decided to investigate the possibility that MTX-based chemotherapy would sensitize the tumors to combination immunotherapy targeting CTLA-4 and PD-1. For this, MCA205 fibrosarcoma-bearing mice received MTX-based chemotherapy alone or in combination with fasting and CRMs (HC or spermidine), followed by optional treatment with CTLA-4/PD-1-blocking antibodies from day 8 post-chemotherapy (FIG. 1A, FIG. 3A, FIG. 4A). Of note, MCA205 fibrosarcomas pretreated with PBS, starvation, HC or spermidine alone (without MTX) did not respond to CTLA-4/PD-1-blockade at all (Data not shown). However, MTX-pretreated tumors responded to immunotherapy leading to complete cure of a significant fraction of mice (3 out of 10). This fraction increased when the MTX pre-treatment was associated with starvation (7 out of 10 tumor-free mice), HC (7 out of 10 tumor-free mice) or spermidine (8 out of 10 tumor-free mice) (FIG. 6A-C). Upon pretreatment with MTX plus spermidine, PD-1 blockade alone was as efficient as the combination therapy targeting both PD-1 and CTLA-4, while CTLA-4 blockade alone failed to cure mice (Data not shown).

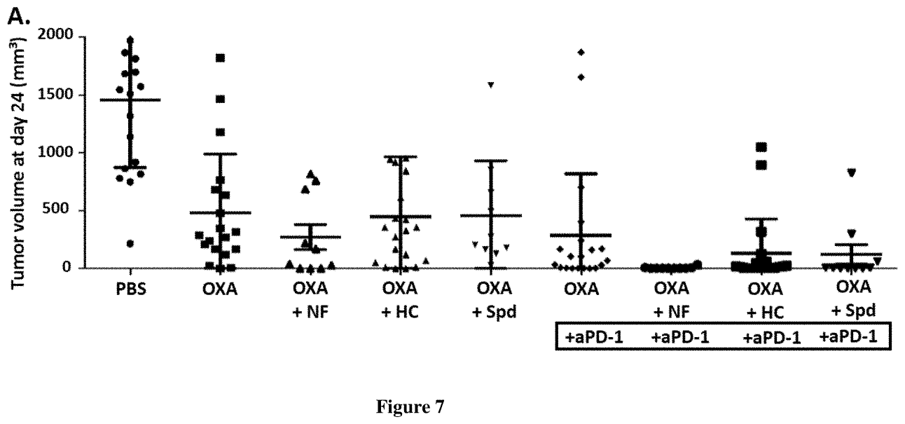

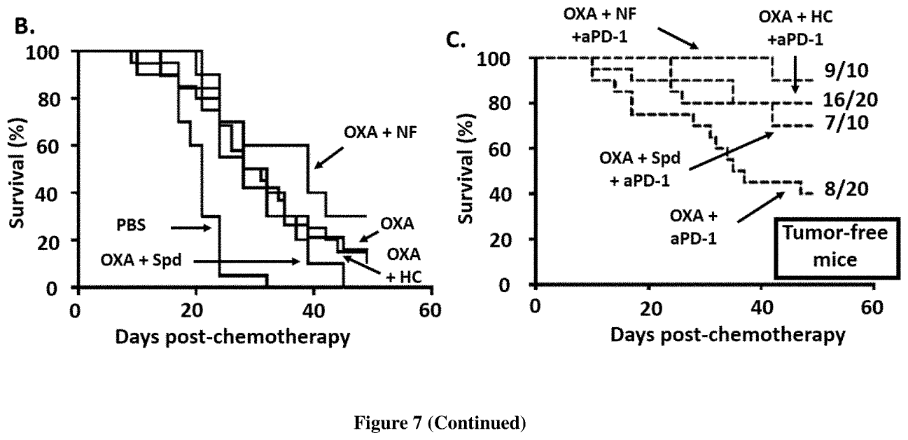

Rather similar results were obtained when MTX was replaced by another chemotherapeutic agent, oxaliplatin (OXA). Again, OXA alone sensitized to immunotherapy targeting PD-1 alone (without CTLA-4 blockade) and led to complete tumor regression in 8 out 20 fibrosarcoma-bearing mice. This cure rate increased from 40% (OXA+PD-1 blockade) to 90% (9 out of 10 mice), 80% (16 out of 20 mice) and 70% (7 out of 10 mice) when fasting, HC and spermidine, respectively, were added to the therapeutic regimen (FIG. 7A-C). Cancer-free mice failed to develop tumors when rechallenged with the cancer cell type from that they had been cured (MCA205), yet allowed for the growth of an antigenically different malignancy (TC1 non-small cell lung cancers) (FIG. 4G). This observation reflects the induction of a potent cytotoxic T cell response together with the establishment of long-lasting cancer-specific immune memory.

Altogether, these results demonstrate that chemotherapeutics (such as MTX or OXA) sensitize to immunotherapy targeting the PD-1/PD-L1 interaction and that this sensitization effect can be amplified by starvation or CRMs.

### Example 3

Further in vivo experiments are undergoing as hereinafter detailed.

In vivo experimentations. Tumor engraftment was performed through subcutaneous/orthotopic injection of XMCA205/MC38/PC3/TC1 tumor cells (in 100 μl PBS) in the right flank/orthotopic place of the mice. Tumor volume was monitored using a digital caliper and calculated according to the formula: volume=length×width×height/8×4/3 pi or by adequate imaging model (CT scan, PET scan, fluorescence imaging). When tumor reached 20 mm³ on average, mice underwent fasting (48 hours without food but ad libitum access to water) or were given caloric restriction mimetics (CRMs) such as, hydroxycitrate (HC; 5 mg/ml in drinking water daily), or were treated with mitoxantrone (MTX; 5.17 mg/kg i.p. in 200 μl PBS) Oxaliplatin, carboplatin+pemetrexed, Oxaliplatin+5 FU, or paclitaxel/Nab paclitaxel, or with the immune checkpoint inhibitors (ICIs) anti-PD-1 (10 mg/kg i.p. in 200 μl PBS) and/or anti-CTLA-4 (5 mg/kg i.p. in 200 μl PBS) or or IDO antagonist or VISTA antagonist or TIM3 antagonist or LAG3 antagonist. Tumor size was carefully monitored up to 50 days post-MTX/Chemo. Antitumor immunity induced by the treatment in cured mice was challenged by subcutaneously re-engrafting the same tumor (syngeneic MCA205/MC38/PC3/TC1 cells) in one flank while an antigenically unrelated cancer (3×10⁵ syngeneic TC1/MCA205 or other cells) was implanted into the contralateral flank.

| | lung model TC1 | Colorectal cancer | Pancreatic cancer | Fibrosarcoma |
|---|---|---|---|---|
| | orthotopic | MC38 s.c. | PC3 S s.c. | MCA205 s.c. |
| CRM | Hydroxycitrate | Hydroxycitrate | Hydroxycitrate | Hydroxycitrate |
| Chemotherapy | Oxaliplatin (OXA) or | Oxaliplatin + 5FU = | nab-paclitaxel + | Mitoxantrone |
| | “carboplatin + | folfox : 2^{nd} line) | gemcitabine | |
| | pemetrexed” | | (2^{nd} line human) | |
| | (1st line human) | | | |
| ICI | PD1 inhibitor | PD1 inhibitor | PD1 inhibitor | PD1 inhibitor or IDO antagonist |
| | | | | or VISTA antagonist or TIM3 |
| | | | | antagonist or LAG3 antagonist |
| Groups | Ctrl + ICI seul + | Ctrl + ICI seul + | Ctrl + ICI seul + | Ctrl + ICI seul |
| | CRM/chemotherapy | CRM/chemotherapy + | CRM/chemotherapy + | (PD1 inhibitor or IDO |
| | (OXA or “carboplatin + | CRM/Immuno + | CRM/Immuno + | antagonist or VISTA antagonist |
| | pemetrexed”) + | chemotherapy/ICI + | Chimio/ICI + | or TIM3 antagonist or LAG3 antagonist) + |
| | CRM/Immuno + | CRM/Immuno/Chimio | CRM/Immuno/Chimio | CRM/chemotherapy + CRM/ICI |
| | chemotherapy/ICI | | | (PD1 inhibitor or IDO |
| | (OXA or “carboplatin + | | | antagonist or VISTA antagonist |
| | pemetrexed”) + | | | or TIM3 antagonist or LAG3 antagonist) |
| | CRM/Immuno/Chimio | | | chemotherapy/ICI |
| | (OXA or “carboplatin + | | | (PD1 inhibitor or IDO |
| | pemetrexed”) + | | | antagonist or VISTA antagonist |
| | | | | or TIM3 antagonist or LAG3 antagonist) |
| | | | | CRM/Immuno/chemotherapy |
| | | | | (PD1 inhibitor or IDO |
| | | | | antagonist or VISTA antagonist |
| | | | | or TIM3 antagonist or LAG3 antagonist) |

### Example 4

A multicenter, 3-arms, randomized, double-blind, placebo-controlled Phase II is designed to evaluate the clinical impact of caloric restriction mimetics (hydroxycitrate (HC)±alpha-lipoic acid (ALA) in metastatic non-squamous non-small cell lung cancer (NSLCC) treated with pembrolizumab, carboplatin and pemetrexed.

The proposed placebo control design is both required and appropriate considering that i) the use of placebo group is the most rigorous way for evaluating a treatment efficacy; 2) the placebo will be compared against study drugs added on to standard of care treatment. Therefore, the true added benefit (or risk) of the study drugs will be properly evaluated with no loss of chances for enrolled patients.

Randomization will be performed by means of an integrated interactive voice-response and Web-response system and stratified according to center.

An independent data monitoring committee to assess potential toxicities of HC and ALA will be implemented once 20 patients per group will have achieved the 3 month post inclusion period to discuss corrective measures or study termination in case of toxicities

Patient compliance to HC and ALA per os treatment will be monitored by counting the amount of pills left in their pill organizer.

Patient Follow-Up and Assessments

Randomised patients will be followed-up as per standard clinical practice (i.e. no additional exams).

Quality of life questionnaires (QLQ-C30) will be completed at baseline, M3, M6 and End of treatment.

Biological samples collection will be performed at baseline and M3 (end of chemotherapy) for all randomised patients: de novo tumor biopsies and blood, urine and stool samples.

All eligible patients are to be treated with pembrolizumab (200 mg)+carboplatin (AUC 5 mg/mL)+pemetrexed (500 mg/m²), intravenously every 3 weeks for 4 cycles followed by pembrolizumab (200 mg)+pemetrexed (500 mg/m²) and randomised (1:1:1) to receive:
Arm A: alpha-lipoic acid (ALA, 600 mg 3x/j, per os, morning noon and evening)+hydroxycitrate (HC, dose 800 mg×3/j, per os, morning noon and evening)
Arm B: HC (+ALA matching placebo)
Arm C: matching placebos.

### CONCLUSION

The complete and permanent cure of cancer is a close-to-utopian goal. Full anti-neoplastic efficacy is even difficult to be achieved in rodent models. Here, we provide evidence that a combination of chemotherapy with fasting or CRMs sensitizes tumor-bearing mice to immunotherapy, hence allowing to achieve a durable disappearance of macroscopic cancers. Such a combination therapy (fasting or CRM plus chemotherapy plus immunotherapy) led to permanent disappearance of established cancers in a sizable fraction of the treated mice, causing the induction of a protective anticancer immune response.

### REFERENCES

Throughout this application, various references describe the state of the art to which this invention pertains. The disclosures of these references are hereby incorporated by reference into the present disclosure.

1. F. Madeo, F. Pietrocola, T. Eisenberg, G. Kroemer, Caloric restriction mimetics: towards a molecular definition. Nat Rev Drug Discov 13, 727-740 (2014).

2. G. Marino, F. Pietrocola, T. Eisenberg, Y. Kong, S. A. Malik, A. Andryushkova, S. Schroeder, T. Pendl, A. Harger, M. Niso-Santano, N. Zamzami, M. Scoazec, S. Durand, D. P. Enot, A. F. Fernandez, I. Martins, O. Kepp, L. Senovilla, C. Bauvy, E. Morselli, E. Vacchelli, M. Bennetzen, C. Magnes, F. Sinner, T. Pieber, C. Lopez-Otin, M. C. Maiuri, P. Codogno, J. S. Andersen, J. A. Hill, F. Madeo, G. Kroemer, Regulation of autophagy by cytosolic acetyl-coenzyme A. Mol Cell 53, 710-725 (2014).

3. F. Pietrocola, F. Castoldi, M. Markaki, S. Lachkar, G. Chen, D. Enot, S. Durand, N. Bossut, M. Tong, S. Malik, F. Loos, N. Dupont, G. Marino, N. Abdelkader, F. Madeo, M. Maiuri, R. Kroemer, P. Codogno, J. Sadoshima, N. Tavernarakis, G. Kroemer, Aspirin Recapitulates Features of Caloric Restriction. Cell Rep, (2018).

4. F. Pietrocola, S. Lachkar, D. P. Enot, M. Niso-Santano, J. M. Bravo-San Pedro, V. Sica, V. Izzo, M. C. Maiuri, F. Madeo, G. Marino, G. Kroemer, Spermidine induces autophagy by inhibiting the acetyltransferase EP300. Cell Death Differ 22, 509-516 (2015).

5. M. Lakshminarasimhan, D. Rauh, M. Schutkowski, C. Steegborn, Sirtl activation by resveratrol is substrate sequence-selective. Aging (Albany NY) 5, 151-154 (2013).

6. M. Gertz, G. T. Nguyen, F. Fischer, B. Suenkel, C. Schlicker, B. Franzel, J. Tomaschewski, F. Aladini, C. Becker, D. Wolters, C. Steegborn, A molecular mechanism for direct sirtuin activation by resveratrol. PLoS One 7, e49761 (2012).

7. J. M. Villalba, F. J. Alcain, Sirtuin activators and inhibitors. Biofactors 38, 349-359 (2012).

8. M. T. Borra, B. C. Smith, J. M. Denu, Mechanism of human SIRT1 activation by resveratrol. J Biol Chem 280, 17187-17195 (2005).

9. F. Pietrocola, J. Pol, E. Vacchelli, S. Rao, D. P. Enot, E. E. Baracco, S. Levesque, F. Castoldi, N. Jacquelot, T. Yamazaki, L. Senovilla, G. Marino, F. Aranda, S. Durand, V. Sica, A. Chery, S. Lachkar, V. Sigl, N. Bloy, A. Buque, S. Falzoni, B. Ryffel, L. Apetoh, F. Di Virgilio, F. Madeo, M. C. Maiuri, L. Zitvogel, B. Levine, J. M. Penninger, G. Kroemer, Caloric Restriction Mimetics Enhance Anticancer Immunosurveillance. Cancer Cell 30, 147-160 (2016).

10. L. Galluzzi, A. Buque, O. Kepp, L. Zitvogel, G. Kroemer, Immunogenic cell death in cancer and infectious disease. Nat Rev Immunol 17, 97-111 (2017).

11. N. Bloy, P. Garcia, C. M. Laumont, J. M. Pitt, A. Sistigu, G. Stoll, T. Yamazaki, E. Bonneil, A. Buque, J. Humeau, J. W. Drijfhout, G. Meurice, S. Walter, J. Fritsche, T. Weinschenk, H. G. Rammensee, C. Melief, P. Thibault, C. Perreault, J. Pol, L. Zitvogel, L. Senovilla, G. Kroemer, Immunogenic stress and death of cancer cells: Contribution of antigenicity vs adjuvanticity to immunosurveillance. Immunol Rev 280, 165-174 (2017).

12. E. Vacchelli, Y. Ma, E. E. Baracco, A. Sistigu, D. P. Enot, F. Pietrocola, H. Yang, S. Adjemian, K. Chaba, M. Semeraro, M. Signore, A. De Ninno, V. Lucarini, F. Peschiaroli, L. Businaro, A. Gerardino, G. Manic, T. Ulas, P. Gunther, J. L. Schultze, O. Kepp, G. Stoll, C. Lefebvre, C. Mulot, F. Castoldi, S. Rusakiewicz, S. Ladoire, L. Apetoh, J. M. Bravo-San Pedro, M. Lucattelli, C. Delarasse, V. Boige, M. Ducreux, S. Delaloge, C. Borg, F. Andre, G. Schiavoni, I. Vitale, P. Laurent-Puig, F. Mattei, L. Zitvogel, G. Kroemer, Chemotherapy-induced antitumor immunity requires formyl peptide receptor 1. Science 350, 972-978 (2015).

13. A. Sistigu, T. Yamazaki, E. Vacchelli, K. Chaba, D. P. Enot, J. Adam, I. Vitale, A. Goubar, E. E. Baracco, C. Remedios, L. Fend, D. Hannani, L. Aymeric, Y. Ma, M. Niso-Santano, O. Kepp, J. L. Schultze, T. Tuting, F. Belardelli, L. Bracci, V. La Sorsa, G. Ziccheddu, P. Sestili, F. Urbani, M. Delorenzi, M. Lacroix-Triki, V. Quidville, R. Conforti, J. P. Spano, L. Pusztai, V. Poirier-Colame, S. Delaloge, F. Penault-Llorca, S. Ladoire, L. Arnould, J. Cyrta, M. C. Dessoliers, A. Eggermont, M. E. Bianchi, M. Pittet, C. Engblom, C. Pfirschke, X. Preville, G. Uze, R. D. Schreiber, M. T. Chow, M. J. Smyth, E. Proietti, F. Andre, G. Kroemer, L. Zitvogel, Cancer cell-autonomous contribution of type I interferon signaling to the efficacy of chemotherapy. Nat Med 20, 1301-1309 (2014).

14. M. Michaud, I. Martins, A. Q. Sukkurwala, S. Adjemian, Y. Ma, P. Pellegatti, S. Shen, O. Kepp, M. Scoazec, G. Mignot, S. Rello-Varona, M. Tailler, L. Menger, E. Vacchelli, L. Galluzzi, F. Ghiringhelli, F. di Virgilio, L. Zitvogel, G. Kroemer, Autophagy-dependent anticancer immune responses induced by chemotherapeutic agents in mice. Science 334, 1573-1577 (2011).

15. G. Kroemer, L. Galluzzi, O. Kepp, L. Zitvogel, Immunogenic cell death in cancer therapy. Annu Rev Immunol 31, 51-72 (2013).

16. Y. Ma, L. Aymeric, C. Locher, S. R. Mattarollo, N. F. Delahaye, P. Pereira, L. Boucontet, L. Apetoh, F. Ghiringhelli, N. Casares, J. J. Lasarte, G. Matsuzaki, K. Ikuta, B. Ryffel, K. Benlagha, A. Tesniere, N. Ibrahim, J. Dechanet-Merville, N. Chaput, M. J. Smyth, G. Kroemer, L. Zitvogel, Contribution of IL-17-producing gamma delta T cells to the efficacy of anticancer chemotherapy. J Exp Med 208, 491-503 (2011).

17. F. Ghiringhelli, L. Apetoh, A. Tesniere, L. Aymeric, Y. Ma, C. Ortiz, K. Vermaelen, T. Panaretakis, G. Mignot, E. Ullrich, J. L. Perfettini, F. Schlemmer, E. Tasdemir, M. Uhl, P. Genin, A. Civas, B. Ryffel, J. Kanellopoulos, J. Tschopp, F. Andre, R. Lidereau, N. M. McLaughlin, N. M. Haynes, M. J. Smyth, G. Kroemer, L. Zitvogel, Activation of the NLRP3 inflammasome in dendritic cells induces IL-1beta-dependent adaptive immunity against tumors. Nat Med 15, 1170-1178 (2009).

18. L. Apetoh, F. Ghiringhelli, A. Tesniere, M. Obeid, C. Ortiz, A. Criollo, G. Mignot, M. C. Maiuri, E. Ullrich, P. Saulnier, H. Yang, S. Amigorena, B. Ryffel, F. J. Barrat, P. Saftig, F. Levi, R. Lidereau, C. Nogues, J. P. Mira, A. Chompret, V. Joulin, F. Clavel-Chapelon, J. Bourhis, F. Andre, S. Delaloge, T. Tursz, G. Kroemer, L. Zitvogel, Toll-like receptor 4-dependent contribution of the immune system to anticancer chemotherapy and radiotherapy. Nat Med 13, 1050-1059 (2007).

19. M. Obeid, A. Tesniere, F. Ghiringhelli, G. M. Fimia, L. Apetoh, J. L. Perfettini, M. Castedo, G. Mignot, T. Panaretakis, N. Casares, D. Metivier, N. Larochette, P. van Endert, F. Ciccosanti, M. Piacentini, L. Zitvogel, G. Kroemer, Calreticulin exposure dictates the immunogenicity of cancer cell death. Nat Med 13, 54-61 (2007).

20. W. H. Fridman, L. Zitvogel, C. Sautes-Fridman, G. Kroemer, The immune contexture in cancer prognosis and treatment. Nat Rev Clin Oncol 14, 717-734 (2017).

21. J. M. Pitt, G. Kroemer, L. Zitvogel, Immunogenic and Non-immunogenic Cell Death in the Tumor Microenvironment. Adv Exp Med Biol 1036, 65-79 (2017).

22. C. Pfirschke, C. Engblom, S. Rickelt, V. Cortez-Retamozo, C. Garris, F., Pucci, T. Yamazaki, V. Poirier-Colame, A. Newton, Y. Redouane, Y. J. Lin, G. Wojtkiewicz, Y. Iwamoto, M. Mino-Kenudson, T. G. Huynh, R. O. Hynes, G. J. Freeman, G. Kroemer, L. Zitvogel, R. Weissleder, M. J. Pittet, Immunogenic Chemotherapy Sensitizes Tumors to Checkpoint Blockade Therapy. Immunity 44, 343-354 (2016).

23. E. B. Golden, L. Apetoh, Radiotherapy and immunogenic cell death. Semin Radiat Oncol 25, 11-17 (2015).

24. R. W. Jenkins, D. A. Barbie, K. T. Flaherty, Mechanisms of resistance to immune checkpoint inhibitors. Br J Cancer 118, 9-16 (2018).

25. P. Sharma, S. Hu-Lieskovan, J. A. Wargo, A. Ribas, Primary, Adaptive, and Acquired Resistance to Cancer Immunotherapy. Cell 168, 707-723 (2017).

26. J. M. Michot, C. Bigenwald, S. Champiat, M. Collins, F. Carbonnel, S. Postel-Vinay, A. Berdelou, A. Varga, R. Bahleda, A. Hollebecque, C. Massard, A. Fuerea, V. Ribrag, A. Gazzah, J. P. Armand, N. Amellal, E. Angevin, N. Noel, C. Boutros, C. Mateus, C. Robert, J. C. Soria, A. Marabelle, O. Lambotte, Immune-related adverse events with immune checkpoint blockade: a comprehensive review. Eur J Cancer 54, 139-148 (2016).

27. F. Madeo, T. Eisenberg, F. Pietrocola, G. Kroemer, Spermidine in health and disease. Science 359, (2018).

28. T. Eisenberg, M. Abdellatif, S. Schroeder, U. Primessnig, S. Stekovic, T. Pendl, A. Harger, J. Schipke, A. Zimmermann, A. Schmidt, M. Tong, C. Ruckenstuhl, C. Dammbrueck, A. S. Gross, V. Herbst, C. Magnes, G. Trausinger, S. Narath, A. Meinitzer, Z. Hu, A. Kirsch, K. Eller, D. Carmona-Gutierrez, S. Buttner, F. Pietrocola, O. Knittelfelder, E. Schrepfer, P. Rockenfeller, C. Simonini, A. Rahn, M. Horsch, K. Moreth, J. Beckers, H. Fuchs, V. Gailus-Durner, F. Neff, D. Janik, B. Rathkolb, J. Rozman, M. H. de Angelis, T. Moustafa, G. Haemmerle, M. Mayr, P. Willeit, M. von Frieling-Salewsky, B. Pieske, L. Scorrano, T. Pieber, R. Pechlaner, J. Willeit, S. J. Sigrist, W. A. Linke, C. Muhlfeld, J. Sadoshima, J Dengjel, S. Kiechl, G. Kroemer, S. Sedej, F. Madeo, Cardioprotection and lifespan extension by the natural polyamine spermidine. Nat Med 22, 1428-1438 (2016).

1-15. (canceled)

16. A method of treating cancer in a patient in need thereof comprising administering to the patient a therapeutically effective combination of chemotherapy and an immune checkpoint inhibitor with at least one caloric restriction mimetic.

17. The method of claim 16, wherein the at least one caloric restriction mimetic is selected from the group consisting of inhibitors of mitochondrial pyruvate carrier complex (MPC), inhibitors of mitochondrial carnitine palmitoytransferase-1 (CTP1), inhibitors of mitochondrial citrate carrier (CiC), inhibitors of ATP-citrate lyase (ACLY), EP300 acetyltransferase inhibitors, and inhibitors of acyl-CoA synthetase short-chain family member 2 (ACCS2).

18. The method according to claim 16, wherein the at least one caloric restriction mimetic is selected from the group consisting of hydroxycitrate, lipoic acid, spermidine, and mixtures thereof.

19. The method according to claim 16, wherein the at least one caloric restriction mimetic is hydroxycitrate.

20. The method according to claim 16, wherein the at least one caloric restriction mimetic is hydroxycitrate in association with lipoic acid.

21. The method according to claim 16, wherein the chemotherapy consists in administrating to the patient a therapeutically effective amount of a chemotherapeutic agent selected from the group consisting of alkylating agents; alkyl sulfonates; aziridines; ethylenimines; methyl amelamines; acetogenins; a camptothecin; bryostatin; callystatin; CC-1065; cryptophycins; dolastatin; duocarmycin; eleutherobin; pancratistatin; a sarcodictyin; spongistatin; nitrogen mustards; nitrosureas; antibiotics; dynemicin; bisphosphonates; an esperamicin; neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites; folic acid analogs; purine analogs; pyrimidine analogs; androgens; anti-adrenals; folic acid replenisher; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofuran; spirogermanium; tenuazonic acid; triaziquone; 2,2′,2″-trichlorotriethylamine; trichothecenes; urethan; vindesine; dacarbazine; mannomustine; mitobronitol; mitolactol; pipobroman; gacytosine; arabinoside (“Ara-C”); cyclophosphamide; thiotepa; taxoids; chlorambucil; gemcitabine; 6-thioguanine; mercaptopurine; methotrexate; platinum coordination complexes; vinblastine; platinum; etoposide (VP-16); ifosfamide; vincristine; vinorelbine; novantrone; teniposide; edatrexate; daunomycin; aminopterin; xeloda; ibandronate; irinotecan; topoisomerase inhibitor RFS 2000; difluoromethylomithine (DMFO); retinoids; capecitabine; and pharmaceutically acceptable salts, acids or derivatives of any of the above.

22. The method according to claim 16, wherein the chemotherapy consists in administrating to the patient a therapeutically effective amount of a chemotherapeutic agent selected from the group consisting of cyclophosphamide, dolastatin, pancratistatin, mechlorethamine, bleomycins, dactinomycin, daunorubicin, doxorubicin, morpholino-doxorubicin, cyanomorpholino-doxorubicin, 2-pyrrolino-doxorubicin, deoxy doxorubicin, epirubicin, idarubicin, 5-fluorouracil, trimetrexate, epothilones, lonidamine, maytansine, mitoxantrone, PSK polysaccharide complex, verrucarin A, vindesine, cytosine arabinoside, paclitaxel, nab-paclitaxel, docetaxel, 6-thioguanine, cisplatin, oxaliplatin, carboplatin, vinblastine, platinum, ansamitocins, vincristine, vinorelbine, novantrone, daunomycin, irinotecan, retinoic acid, bortezomib, digitoxin, digoxin, patupilone, hypericin, cetuximab, septacidin, hedamycin, CDDP, mitomycin C, temozolomide, gemcitabine, and pemetrexed.

23. The method according to claim 16, wherein the immune checkpoint inhibitor is selected from the group consisting of PD-1 antagonists, PD-L1 antagonists, PD-L2 antagonists, CTLA-4 antagonists, VISTA antagonists, TIM-3 antagonists, LAG-3 antagonists, IDO antagonists, KIR2D antagonists, A2AR antagonists, B7-H3 antagonists, B7-H4 antagonists, and BTLA antagonists.

24. The method according to claim 16, wherein the immune checkpoint inhibitor is a therapeutically effective combination of a PD-1 antagonist with a CTLA-4 antagonist.

25. The method according to claim 16, wherein the immune checkpoint inhibitor is selected from the group consisting of nivolumab, pembrolizumab, pidilizumab, avelumab, durvalumab, atezolimumab, ipilimumab, and tremelimumab.

26. The method according to claim 16, wherein:
the at least one caloric restriction mimetic is hydroxycitrate or hydroxycitrate in association with lipoic acid;
the chemotherapy consists in administrating to the patient a therapeutically effective amount of a chemotherapeutic agent selected from cisplatin, oxaliplatin, carboplatin; taxanes selected from paclitaxel, nab-paclitaxel, docetaxel, and taxotere; vinca alkaloids selected from vindesine, vinblastine, vincristine, and vinorelbine; anthracyclines selected from mitoxantrone, daunorubicin, doxorubicin, epirubicin, idarubicin, valrubicin, and ditrisarubicin; gemcitabine; pemetrexed; mixtures thereof and pharmaceutically acceptable salts thereof; and
the immune checkpoint inhibitor is selected from the group consisting of nivolumab, pembrolizumab, pidilizumab, avelumab, durvalumab atezolimumab, ipilimumab, and tremelimumab.

27. The method according to claim 16, wherein:
the at least one caloric restriction mimetic is hydroxycitrate or hydroxycitrate in association with lipoic acid;
the chemotherapy consists in administrating to the patient a therapeutically effective amount of a chemotherapeutic agent selected from cisplatin, oxaliplatin and carboplatin; or a simultaneous or sequential administration of carboplatin and pemetrexed; or a simultaneous or sequential administration of oxaliplatin and 5-FU; taxanes selected from paclitaxel, nab-paclitaxel, docetaxel, and taxotere; gemcitabine; pemetrexed; mitoxantrone; and mixtures thereof and pharmaceutically acceptable salts thereof; and
the immune checkpoint inhibitor is selected from the group consisting of nivolumab, pembrolizumab, pidilizumab, avelumab, durvalumab atezolimumab, ipilimumab and tremelimumab.

28. The method according to claim 16, wherein the immune checkpoint inhibitor is administrated simultaneously with the chemotherapy and/or the immune checkpoint inhibitor.

29. The method according to claim 16, wherein the immune checkpoint inhibitor is administrated prior to the chemotherapy and/or the immune checkpoint inhibitor.

30. The method of claim 16, wherein the cancer is selected from the group consisting of neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; bronchioloalveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulated sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

31. The method of claim 16, wherein the cancer is selected from the group consisting of adenocarcinoma, lung cancer, pancreas carcinoma, stomach carcinoma, colon carcinoma, rectal carcinoma, glioma, and glioblastoma.
